(19) United States
(12) Patent Application Publication
(10) Pub. No.: US 2019/0292256 A1
(43) Pub. Date: Sep. 26, 2019

(54) Compounds and Methods to Measure Metabolic Function and Restore Normal Metabolic Function

(71) Applicants: Kenneth Walsh, Carlisle, MA (US); Noriyuki Ouchi, Mizuho-ku (JP)

(72) Inventors: Kenneth Walsh, Carlisle, MA (US); Noriyuki Ouchi, Mizuho-ku (JP)

(21) Appl. No.: 16/259,150

(22) Filed: Jan. 28, 2019

Related U.S. Application Data
(63) Continuation of application No. 15/267,712, filed on Sep. 16, 2016, now abandoned.
Application No. 15/267,712 is a division of application No. 13/100,895, filed on May 4, 2011, now abandoned.
(60) Provisional application No. 61/332,051, filed on May 6, 2010.

(51) Int. Cl. C07K 16/28; C07K 16/18; A61K 38/17

(52) U.S. Cl. CPC C07K 16/28; C07K 16/18; C07K 16/2896; G01N 2800/04; C07K 2317/24; C07K 2317/34; C07K 2317/76; A61K 38/1709

(57) ABSTRACT

The invention relates to treatment to restore normal metabolic function, including but not limited to normal glucose levels. The invention in one embodiment contemplates methods of reducing elevated glucose levels in subjects with elevated glucose levels by administering a composition comprising at least a portion of human Sfrp5. The invention in one embodiment contemplates methods of reducing elevated glucose levels in subjects with elevated glucose levels by administering a composition comprising an inhibitor of Wnt5a, including but not limited to an antibody inhibitor.

### FIELD OF THE INVENTION

The invention relates to compounds and methods (including methods of treatment) to restore normal metabolic function in humans, including but not limited to normal glucose levels. The invention in one embodiment contemplates methods of reducing elevated glucose levels in subjects with elevated glucose levels by administering a composition comprising at least a portion of human Sfrp5. The invention in one embodiment contemplates methods of reducing elevated glucose levels in subjects with elevated glucose levels by administering a composition comprising an inhibitor of Wnt5a, including but not limited to an antibody inhibitor. In addition, the present invention contemplates compounds and methods for detecting and measuring metabolic function.

### BACKGROUND OF THE INVENTION

Obesity is a major health problem that is linked to the development of metabolic disorders that are associated with a low-grade inflammatory state in adipose tissue. It is increasingly recognized that adipose tissue secretes a variety of bioactive substances that are referred to as adipokines [1-3]. The majority of these adipokines are pro-inflammatory including TNFα, IL-6 and leptin. However, the well-studied cytokine adiponectin is anti-inflammatory and it promotes insulin-sensitization and cardio-protection [2, 4]. However, adiponectin cannot be practically used because the amounts needed for therapeutic treatment are too great.

Clearly, other safe and effective treatments are needed for human administration.

### SUMMARY OF THE INVENTION

The invention relates to compositions and methods (including methods of treatment) to restore normal metabolic function in humans, including but not limited to normal glucose levels. The invention in one embodiment contemplates methods of reducing elevated glucose levels in subjects with elevated glucose levels by administering a composition comprising at least a portion of human Sfrp5 (or a nucleic acid construct capable of expressing human Sfrp5). The invention in one embodiment contemplates methods of reducing elevated glucose levels in subjects with elevated glucose levels by administering a composition comprising an inhibitor of Wnt5a, including but not limited to an antibody inhibitor, such as a humanized antibody with affinity for Wnt5a. In addition, the present invention contemplates compounds and methods for detecting and measuring metabolic function.

In one embodiment the invention relates to a host cell comprising an expression vector, said vector encoding human Sfrp5 or a portion thereof. In further embodiments, said host cell is capable of expressing said human Sfrp5 or portion thereof as a soluble protein at a level greater than or equal to 5% of the total cellular protein. In further embodiments, said host cell is capable of expressing said human Sfrp5 or portion thereof as a soluble protein at a level greater than or equal to 15% of the total cellular protein. In further embodiments, said vector encodes a portion consisting of a domain of human Sfrp5. In further embodiments, said vector encodes a fusion protein comprising at least a portion of human Sfrp5, said portion comprising a portion of the sequence of SEQ ID NO: 2. In further embodiments, said fusion protein comprises a poly-histidine tract. In further embodiments, said fusion protein comprises at least a portion of an immunoglobulin molecule. In further embodiments, said portion of an immunoglobulin molecule consists of an Fc fragment.

In another embodiment the invention relates to a soluble fusion protein comprising at least a portion of human Sfrp5, said portion comprising a portion of the sequence of SEQ ID NO: 2. In another embodiment the invention relates to the fusion protein, wherein said portion consists of at least one domain of human Sfrp5. In another embodiment the invention relates to the fusion protein, wherein said fusion protein comprises a poly-histidine tract. In another embodiment the invention relates to the fusion protein, wherein said fusion protein comprises at least a portion of an immunoglobulin molecule. In another embodiment the invention relates to the fusion protein, wherein said portion of an immunoglobulin molecule consists of an Fc fragment. In another embodiment the invention relates to the fusion protein, wherein said fusion protein is substantially endotoxin-free.

In another embodiment the invention relates to a method of reducing elevated glucose levels, comprising: a) providing a subject with elevated glucose levels and a composition comprising at least a portion of human Sfrp5; b) administering said composition to said subject; and c) measuring said glucose levels of said subject until they are reduced. In another embodiment the invention relates to a method of reducing elevated glucose levels, wherein said subject is a human. In another embodiment relates to a method, wherein said elevated glucose levels are reduced by at least 20%. In another embodiment relates to a method, wherein said elevated glucose levels are reduced by at least 40%. In another embodiment relates to a method, wherein said composition comprises a fusion protein comprising at least a portion of human Sfrp5, said portion comprising a portion of the sequence of SEQ ID NO: 2. In another embodiment relates to a method, wherein said portion consists of at least one domain of human Sfrp5. In another embodiment relates to a method, wherein said fusion protein comprises a poly-histidine tract. In another embodiment relates to a method, wherein said fusion protein comprises at least a portion of an immunoglobulin molecule. In another embodiment relates to a method, wherein said portion of an immunoglobulin molecule consists of an Fc fragment.

In another embodiment the invention relates to a method of reducing elevated glucose levels, comprising: a) providing a subject with elevated glucose levels and a composition comprising an expression vector, said vector capable of expressing at least a portion of human Sfrp5 in vivo; b) administering said composition to said subject; and c) measuring said glucose levels of said subject until they are reduced. In another embodiment the invention relates to a method of reducing elevated glucose levels, comprising: a) providing a subject with elevated glucose levels and an implantable device, said device capable of releasing at least a portion of human Sfrp5 in vivo; b) implanting said device in said subject; and c) measuring said glucose levels of said subject until they are reduced.

In another embodiment the invention relates to a method of reducing elevated glucose levels, comprising: a) providing a subject with elevated glucose levels and a composition comprising an antibody (e.g. humanized antibody) or portion thereof reactive with human Wnt5a; b) administering said composition to said subject; and c) measuring said glucose levels of said subject until they are reduced. The present invention also contemplates a composition comprising humanized monoclonal antibody reactive with human Wnt5a.

In another embodiment, the present invention contemplates a method of measuring metabolic function (by measuring markers in tissue or blood, preferably plasma or serum), comprising: providing i) a sample (e.g. tissue, blood, secretion, etc.) from a subject and ii) a reagent (or other means) for measuring human Sfrp5 protein (or fragments thereof) or human Sfrp5 nucleic acid (or portions thereof); measuring the level of Sfrp5 protein or nucleic acid as an indicator of metabolic function. In one embodiment, said reagent is an antibody reactive with human Sfrp5 protein. In another embodiment, said antibody is specific for human Sfrp5 (i.e. not reactive with other human proteins). In another embodiment, said antibody is reactive with human, but unreactive with mouse Sfrp5. The present invention contemplates these antibodies as compositions. In one embodiment, said reagent is an oligonucleotide probe (with a region of complementarity for human Sfrp5 nucleic acid) for measuring human Sfrp5 mRNA. In one embodiment, mRNA is measured in tissue biopsies.

### Definitions

As used herein, the term “fusion protein” refers to a chimeric protein containing the protein of interest (i.e., human Sfrp5 or fragments thereof) joined to an exogenous protein fragment (the fusion partner which consists of another protein or protein fragment). The fusion partner may enhance solubility or half-life of the Sfrp5 protein or protein fragment as expressed in a (preferably human) host cell, and may also provide an affinity tag to allow purification of the recombinant fusion protein from the host cell or culture supernatant, or both. If desired, the fusion protein may be removed from the protein of interest prior to administration by a variety of enzymatic or chemical means known to the art.

As used herein, the term “poly-histidine tract” when used in reference to a fusion protein refers to the presence of two to ten histidine residues at either the amino- or carboxy-terminus of a protein of interest, i.e. Sfrp5 or portion thereof (e.g. a domain). A poly-histidine tract of six to ten residues is preferred. The poly-histidine tract is also defined functionally as being a number of consecutive histidine residues added to the protein of interest which allows the affinity purification of the resulting fusion protein on a nickel-chelate column.

The term “subject” includes humans and non-human animals. In the case of humans, the term includes both in-patients and out-patients, and particularly the elderly and the obese, whether or not under the care of a medical professional.

As used herein “immunoglobulin” refers to any of a group of large glycoproteins that are secreted by plasma cells and that function as antibodies in the immune response by binding with specific antigens. The specific antigen bound by an immunoglobulin may or may not be known. There are five classes of immunoglobulins: IgA, IgD, IgE, IgG, and IgM.

The twin “antibody,” as used herein, is intended to refer to immunoglobulin molecules comprised of four polypeptide chains, two heavy (H) chains and two light (L) chains (lambda or kappa) inter-connected by disulfide bonds. An antibody has a known specific antigen with which it binds. Each heavy chain of an antibody is comprised of a heavy chain variable region (abbreviated herein as HCVR, HV or VH) and a heavy chain constant region. The heavy chain constant region is comprised of three domains, CH1, CH2 and CH3. Each light chain is comprised of a light chain variable region (abbreviated herein as LCVR or VL or KV or LV to designate kappa or lambda light chains) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each variable region (VH or VL) contains 3 CDRs, designated CDR1, CDR2 and CDR3. Each variable region also contains 4 framework sub-regions, designated FR1, FR2, FR3 and FR4.

As used herein, the term “antibody fragments” refers to a portion of an intact antibody. Examples of antibody fragments include, but are not limited to, linear antibodies, single-chain antibody molecules, Fv, Fab and F(ab′)₂ fragments, and multispecific antibodies formed from antibody fragments. The antibody fragments preferably retain at least part of the heavy and/or light chain variable region.

As used herein, “humanized” forms of non-human (e.g., murine) antibodies are antibodies that contain minimal (e.g. less tan 10%) sequence, or no sequence, derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are generally made to further refine antibody performance. In general, the humanized antibody will comprise substantially all of at least one, and typically two, variable domains, in which all or substantially all of the hypervariable loops correspond to those of a nonhuman immunoglobulin and all or substantially all of the FR residues are those of a human immunoglobulin sequence. The humanized antibody may also comprise at least a portion of an immunoglobulin constant region (Fc), typically that of a human immunoglobulin. Examples of methods used to generate humanized antibodies are described in U.S. Pat. No. 5,225,539 to Winter et al. (herein incorporated by reference) [5].

Importantly, early methods for humanizing antibodies often resulted in antibodies with lower affinity than the non-human antibody starting material. More recent approaches to humanizing antibodies address this problem by making changes to the CDRs. See U.S. Patent Application Publication No. 20040162413, hereby incorporated by reference. In some embodiments, the present invention provides an optimized heteromeric variable region (e.g. that may or may not be part of a full antibody other molecule) having equal or higher antigen binding affinity than a donor heteromeric variable region, wherein the donor heteromeric variable region comprises three light chain donor CDRs, and wherein the optimized heteromeric variable region comprises: a) a light chain altered variable region comprising; i) four unvaried human germline light chain framework regions, and ii) three light chain altered variable region CDRs, wherein at least one of the three light chain altered variable region CDRs is a light chain donor CDR variant, and wherein the light chain donor CDR variant comprises a different amino acid at only one, two, three or four positions compared to one of the three light chain donor CDRs (e.g. the at least one light chain donor CDR variant is identical to one of the light chain donor CDRs except for one, two, three or four amino acid differences).

The binding “affinity” is directly related to the ratio of the off-rate constant (generally reported in units of inverse time, e.g., seconds.sup.-1) to the on-rate constant (generally reported in units of concentration per unit time, e.g., molar/second). The binding affinity may be determined by, for example, an ELISA assay, kinetic exclusion assay or surface plasmon resonance. Qualitatively, an antibody with affinity for a protein such as Wnt5a demonstrates higher levels of binding as compared to binding to other proteins.

### DESCRIPTION OF THE FIGURES

FIG. 1 show expression and regulation of Sfrp5 in white adipose tissue. (A) Tissue distribution of Sfrp5 mRNA in wild-type (WT) mice fed normal diet. Sfrp5 mRNA levels were analyzed by quantitative real-time PCR (QRT-PCR) and expressed relative to 18S levels (n=3). WAT, white adipose tissue; BAT, brown adipose tissue; S. Muscle, skeletal muscle. (B) Sfrp5 and Wnt5a transcript levels in adipocyte and stromal vascular (SV) fractions isolated from white adipose tissues of WT lean mice fed a normal diet as measured by QRT-PCR analysis and expressed relative to 36B4 (n=3), (C and D) Expression of Sfrp5 in epididymal fat tissue in lean and obese mice. WT mice at the age of 10 weeks were fed normal diet (ND) or high-fat/high sucrose (HF/HS) diet for 24 weeks (D). Sfrp5 transcript levels in WT and ob/ob mice at the age of 20 weeks (C) and WT mice fed ND or HF/HS diet for 24 weeks (D) were measured by QRT-PCR analysis and expressed relative to 36B4 (n=6-7). Expression of Sfrp5 and Wnt5a protein was determined by immunoblot analysis.

FIG. 2 shows that Sfrp5-deficiency exacerbates metabolic dysfunction in mice fed a high-fat/high sucrose (HF/HS) diet. Sfrp5−/− (KO) and wild-type (WT) mice were fed a normal chow or a HF/HS diet for 12 weeks. (A and B) Glucose tolerance test (A) and insulin tolerance test (B) (n=9 in each group). *, P<0.01 vs. corresponding WT mice. (C) Histological sections of oil red O-stained liver from the HF/HS-fed WT and KO mice. Scale bars=100 μm. (D) Triglyceride (TG) content of liver from HF/HS diet-fed WT and KO mice (n=6). (E) Histological analysis of H&E-stained epididymal white adipose tissue of the HF/HS-fed WT and KO mice. Scale bars=100 μm. Adipocyte cross-sectional areas were determined using Image J program (n=7). (F) Macrophage accumulation in epididymal adipose tissues in WT and KO mice when fed a HF/HS diet. Histological sections were stained with anti-F4/80 antibody. Macrophage infiltration was determined as the number of F4/80-positive cells per mm2 (n=8).

FIG. 3 show the enhancement of JNK1 activation contributes to diet-induced metabolic dysfunction in Sfrp5-deficient mice and Wnt5a-mediated cell activation in vitro. Sfrp5^{-/-} (KO) and wild-type (WT) mice were maintained on a high-fat/high sucrose (HF/HS) diet for 12 weeks. (A) Phosphorylation of JNK (Thr183/Tyr185), cJUN (Ser63) and IRS-1 (Ser307) in fat tissue of WT and KO mice as determined by immunoblots analysis. (B) Akt phosphorylation in adipose tissues of WT and KO mice following insulin administration. (C) Effect of Sfrp5 on Wnt5a-stimulated JNK phosphorylation in adipocytes. 3T3-L1 adipocytes were transduced with adenovirus (Ad) TRE-β-gal or AdTRE-Sfrp5 in the presence of AdCMV-tTA followed by treatment with Wnt5a or vehicle for 30 min (D and E) Effect of the conditioned media from Sfrp5-transfected adipocytes on Wnt5a-induced JNK activation (D) and cytokine expression (E) in macrophages. Peritoneal macrophages were stimulated with Wnt5a or vehicle for 30 min (D) or 24 h (E) in the presence of the conditioned media from 3T3-L1 adipocyte transduced with AdTRE-β-gal or AdTRE-Sfrp5 along with AdCMV-tTA. Transcript levels of TNFα and IL-6 were quantified by QRT-PCR (n=4). (F and G) Contribution of JNK1 to severe insulin resistance caused by Sfrp5-deficiency. WT, Sfrp5^{-/-} (Sfrp5-KO), Jnk1^{-/-} (Jnk1-KO) and Sfrp5^{-/-} Jnk1^{-/-} (Sfrp5/Jnk1-DKO) mice were maintained on a high-fat/high sucrose (HF/HS) diet for 12 weeks. Glucose tolerance test (F) and insulin tolerance test (G) were performed (n=6-7 in each group). *, P<0.01 vs. WT mice. **, P<0.01 vs. Sfrp5-KO mice.

FIG. 4 shows the systemic delivery of Sfrp5 is protective against metabolic dysfunction in obese mice. (A to F) AdTRE-β-gal and AdTRE-Sfrp5 along with AdCMV-tTA, or Ad-adiponectin (APN) were intravenously administered to ob/ob mice at the ages of 20 weeks. (A and B) At 2 weeks after supplementation of adenoviral reagents (β-gal, Sfrp5 or APN), glucose tolerance test (A) and insulin tolerance test (B) were performed (n=5-6 in each group). *, P<0.01 vs. β-gal treatment. **, P<0.05 vs. β-gal treatment. (C) Gene expression of cytokines, chemokine and macrophage markers in epididymal fat tissue from ob/ob mice at 2 weeks after treatment with β-gal or Sfrp5 as quantified by QRT-PCR (n=5). *, P<0.01 vs. β-gal treatment. (D) Phosphorylation of JNK in adipose tissue of ob/ob mice at 2 weeks after treatment with β-gal or Sfrp5. (E and F) Representative histological sections of fat pads stained with H&E (E) and liver stained with oil red O (F) in β-gal- or Sfrp5-treated-ob/ob mice. Scale bars=100 μm. Right panel in F shows quantification of adipocyte size (n=6). Right panel in F shows triglyceride (TG) content of liver (n=6). (G) The adipocyte-secreted factor Sfrp5 protects against metabolic dysfunction by suppressing Wnt5a-induced JNK induction and macrophage activation in a paracrine manner and by reducing Wnt5a-stimulated JNK activation in adipocytes in an autocrine manner.

FIG. 5 shows tissue distribution of Sfrp5 protein in WT mice fed normal diet. Equal amounts of proteins were loaded, and Sfrp5 and GAPDH protein levels were determined by immunoblot analysis. WAT, white adipose tissue; BAT, brown adipose tissue; S. Muscle, skeletal muscle.

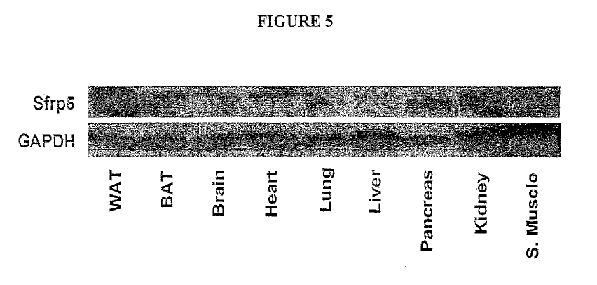

FIG. 6 shows the metabolic parameters and expression of Sfrp5 and Wnt5a in fat tissue in obese Zucker diabetic fatty (ZDF) rats and lean littermates at the age of 12 weeks. (A) Body weight, serum glucose and serum insulin levels in lean and ZDF rats (mean±SEM, n=4). (B) Expression of Sfrp5 and Wnt5a in epididymal fat tissue in lean and ZDF rats. Sfrp5 and TNF [symbol] transcript levels were measured by quantitative real-time PCR (QRT-PCR) analysis and expressed relative to 36B4 (mean±SEM, n=4). Protein expression of Sfrp5 and Wnt5a was determined by immunoblot analysis. Wnt5a/Sfrp5 protein ratio was determined using Image J program.

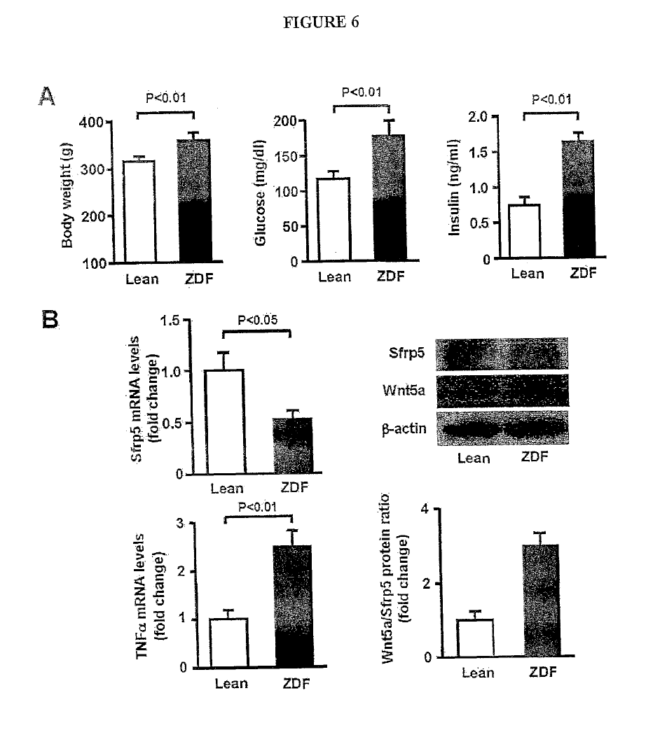

FIG. 7 shows the Expression of Sfrp5 and Wnt5a in epididymal fat tissue in wild-type (WT) mice fed normal diet (ND) or high-fat/high sucrose (HF/HS) diet for 12 weeks. WT mice at the age of 10 weeks were fed ND or HF/HS diet for 12 weeks. Sfrp5 transcript levels were measured by QRT-PCR analysis and expressed relative to 36B4 (n=6-7). Protein expression of Sfrp5 and Wnt5a was determined by immunoblot analysis. Wnt5a/Sfrp5 protein ratio was determined using Image J program.

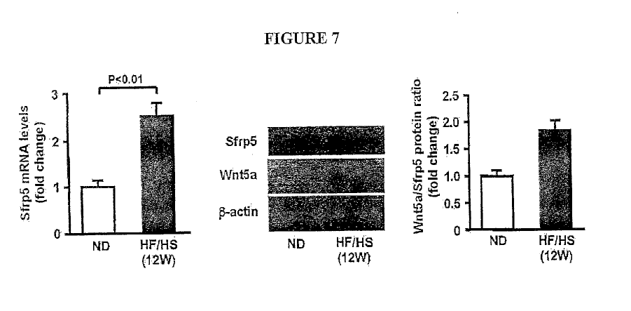

FIG. 8 shows the expression of metabolic parameters and genes in lean and obese mice. (A) Body weight, serum glucose and serum insulin levels in lean and obese mice (mean±SEM, n=5-6). (B) Transcript levels of TNF [symbol] , gp91ₚₕₒₓ, P47ₚₕₒₓ, F4/80, CD68, GRP78 and CHOP in epididymal fat tissue in lean and obese mice. Wild-type mice at the age of 10 week were fed normal diet (ND) for 12 weeks or high-fat/high sucrose (HF/HS) diet for 12 or 24 weeks. Gene expression levels were measured by QRT-PCR analysis and expressed relative to 36B4 (mean±SEM, n=5-6).

FIG. 9 shows the Sfrp5 transcript levels in visceral adipose tissue in human subjects. Expression of Sfrp5 and TNFα, and homeostasis model assessment of insulin resistance (HOMA-IR) were assessed in obese subjects with or without macrophage crown-like structures in visceral fat tissue as determined by immunohistochemical stains with CD68. Transcript levels of Sfrp5 and TNFα were measured by QRT-PCR analysis and expressed relative to 36B4 (mean±SEM, n=18).

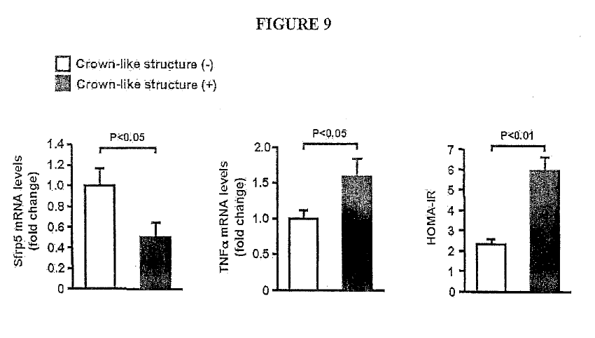

FIG. 10 shows the regulation of Sfrp5 expression in cultured 3T3-L1 adipocytes. (A) Sfrp5 mRNA expression at the different time points during differentiation of 3T3-L1 cells into adipocytes and expressed relative to 18S levels (n=3). *, P<0.01 vs. day 0. (B) Expression of Sfrp5 and adiponectin (APN) in response to various stimuli in adipocytes. Differentiated 3T3-L1 adipocytes were treated with TNFα (10 ng/ml), hydrogen peroxide (H₂O₂, 0.2 mM), tunicamycin (Tun, 5 μg/ml) or vehicle for 24 h. Transcript levels of Sfrp5 and APN were determined by QRT-PCR and expressed relative to 18S levels (mean±SEM, n=3). *, P<0.01 vs. vehicle.

FIG. 11 shows the Sfrp5 protein is ablated in adipose tissue of Sfrp5_{-/-} (KO) mice. Sfrp5 protein expression in epididymal fat tissue of wild-type (WT) and KO mice were assessed by immunoblot analysis.

FIG. 12 shows the body weight, food intake, serum glucose, serum insulin, serum free fatty acid (FFA), serum triglyceride levels, liver weight and fat weight in wild-type (WT) and Sfrp5_{-/-} (KO) mice after 12 weeks of HF/HS diet feeding (mean±SEM, n=9-12).

FIG. 13 shows the expression of macrophage marker, cytokines, chemokine and canonical Wnt-related genes in fat tissues from wild-type (WT) and Sfrp5_{-/-} (KO) mice. (A) Gene expression of F4/80 and CD68 in epididymal adipose tissue from WT and KO mice receiving normal diet (ND) or HF/HS diet was quantified by QRT-PCR and expressed relative to 18S levels (mean±SEM, n=6-7). (B) Gene expression of TNFα, IL-6 and MCP-1 in stromal vascular fraction from epididymal fat tissues of WT and KO mice when fed a normal diet (ND) or HF/HS diet for 12 weeks. Transcript levels were quantified by QRT-PCR and expressed relative to 18S levels (mean±SEM, n=4). (C) Quantification of mRNA levels of CyclinD1 and WISP2 in adipose tissues of WT and KO mice by QRT-PCR methods (mean±SEM, n=9). Gene expression levels were presented relative to 18S levels.

FIG. 14 shows the effect of Sfrp5 on TOPFlash reporter activity and IL-6 expression in adipocytes. (A) Increased production of Sfrp5 in cell lysate and media of 3T3-L1 adipocytes after overexpression of Sfrp5. Differentiated 3T3-L1 adipocytes were transduced with AdTRE-β-gal or AdTRE-Sfrp5 along with AdCMV-tTA. After 48 h of transduction, cells and media were collected, and immunoblot analysis was performed. (B) Effect of Sfrp5 on TOPFlash reporter activity. Differentiated 3T3-L1 adipocytes were transduced with AdTRE-β-gal or AdTRE-Sfrp5 along with AdCMV-tTA for 24 h, and co-transfected with TOPflash and Renilla luciferase control constructs. At 48 h after transduction, cells were treated with Wnt5a (200 ng/ml) or vehicle for 24 h. Reporter activity was analyzed by using dual luciferase assay kit (mean±SEM, n=6). (C) Effect of Sfrp5 on Wnt5a-stimulated IL-6 expression in adipocytes. After 48 h of transduction with adenoviral vectors, cells were treated with Wnt5a (200 ng/ml) or vehicle for 24 h. Transcript levels were quantified by QRT-PCR and expressed relative to 36B4 levels (mean±SEM, n=4). (D) Effect of JNK inhibitor on Wnt5a-induced IL-6 expression in adipocytes. Differentiated 3T3-L1 adipocytes were pretreated with SP600125 (15 μM) or vehicle and stimulated with Wnt5a (200 ng/ml) or vehicle for 24 h. Transcript levels were quantified by QRT-PCR and expressed relative to 36B4 levels (mean±SEM, n=4).

FIG. 15 shows the involvement of JNK in Wnt5a-induced expression of pro-inflammatory cytokines in macrophages. Mouse peritoneal macrophages were pretreated with SP600125 (15 μM) or vehicle and stimulated with Wnt5a (200 ng/ml) or vehicle for 24 h in the presence of the conditioned media from 3T3-L1 adipocyte transduced with AdTRE-β-gal and AdCMV-tTA. Gene expression levels were quantified by QRT-PCR and expressed relative to 36B4 levels (mean±SEM, n=4).

FIG. 16 shows the Body weight and expression of cytokine and chemokine genes of fat tissue in wild-type (WT), Sfrp5_{-/-} (Sfrp5-KO), Jnk1_{-/-} (Jnk1-KO) and Sfrp5_{-/-} (Sfrp5/Jnk1-DKO) mice. (A) Body weight of WT, Sfrp5-KO, Jnk1-KO and Sfrp5/Jnk1-DKO mice maintained on a high-fat/high sucrose (HF/HS) diet for 12 weeks (mean±SEM, n=6-10). (B) Gene expression of TNFα, IL-6 and MCP-1 in epididymal fat tissues from WT, Sfrp5-KO, Jnk1-KO and Sfrp5/Jnk1-DKO mice after 12 weeks of the HF/HS diet feeding. Transcript levels were quantified by QRT-PCR and expressed relative to 18S levels (mean±SEM, n=6-7).

FIG. 17 shows the detection of Sfrp5 in serum of wild-type (WT) mice following adenovirusmediated intravenous injection of Sfrp5. After 10 weeks of HF/HS diet feeding, WT mice were intravenously treated with AdTRE-β-gal (2.5×10₈ pfu total) or AdTRE-Sfrp5 (2.5×10₈ pfu total) along with AdCMV-tTA (2.5×10₈ pfu total). At 1 week after injection of adenoviral vectors (β-gal or Sfrp5), serum was collected. Sfrp5 protein level in serum (10 μl) was determined by immunoblot analysis.

FIG. 18 shows the effect of systemic delivery of Sfrp5 on glucose metabolism in the HF/HS diet-fed wild-type (WT) and Sfrp5−/− (KO) mice. After 10 weeks of HF/HS diet feeding, WT and KO mice were intravenously treated with AdTRE-β-gal (2.5×10⁸ pfu total) or AdTRE-Sfrp5 (2.5×10⁸ pfu total) along with AdCMV-tTA (2.5×10⁸ pfu total). At 2 weeks after injection of adenoviral vectors (β-gal or Sfrp5), glucose tolerance test (A) and insulin tolerance test (B) were performed in the differential experimental groups of mice (mean±SEM, n=6-7 in each group). *, P<0.01 vs. corresponding β-gal treatment.

FIG. 19 shows the human Sfrp5 mRNA levels (fold change), Wnt5a mRNA levels (fold change), Wnt5a/Sfrp5 transcript ratio (fold change), and HOMA index with and without Visceral fat crown-like structure. We compared visceral fat biopsies from obese individuals with inflamed fat (indicated by “crown like structures” of macrophages surrounding dead adipocytes) vs. obese individuals with more normal metabolic properties. The data are excellent and are in line with the rodent data.

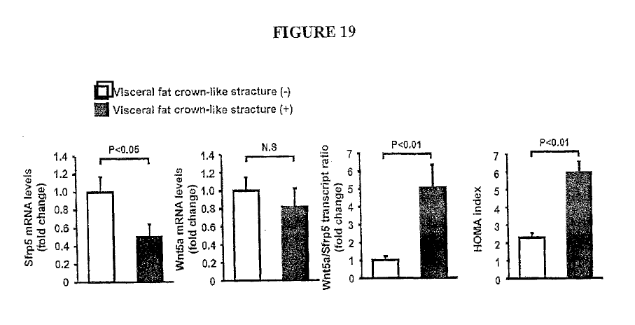

FIG. 20 shows a comparison of human and mouse Sfrp5 amino acid sequences.

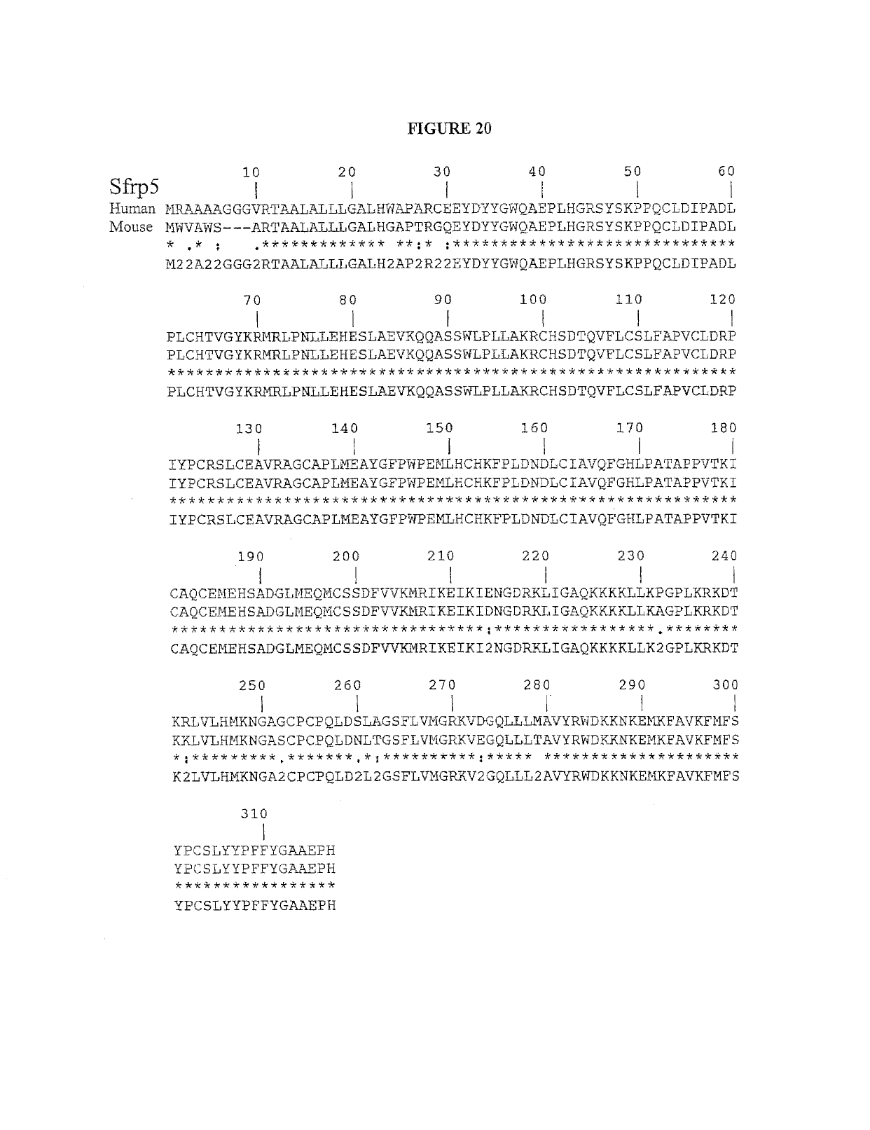

FIG. 21 shows the mouse Sfrp5 amino acid sequence (including the peptide signal) (SEQ ID NO: 1).

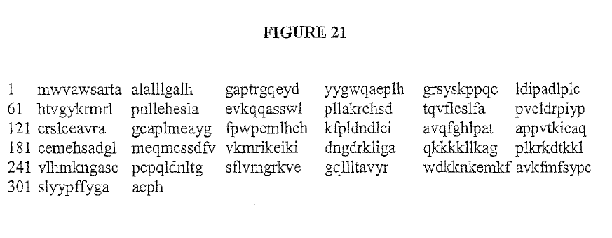

FIG. 22 shows the human Sfrp5 amino acid sequence (including the peptide signal) (SEQ ID NO: 2).

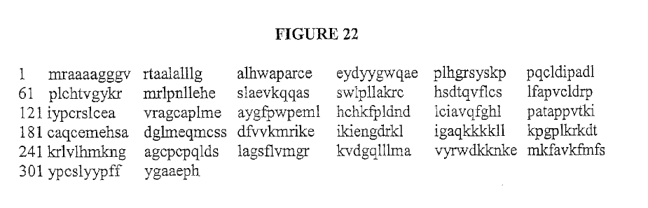

FIG. 23 shows the nucleotide sequence of the nucleic acid encoding full length human Sfrp5 (SEQ ID NO: 3).

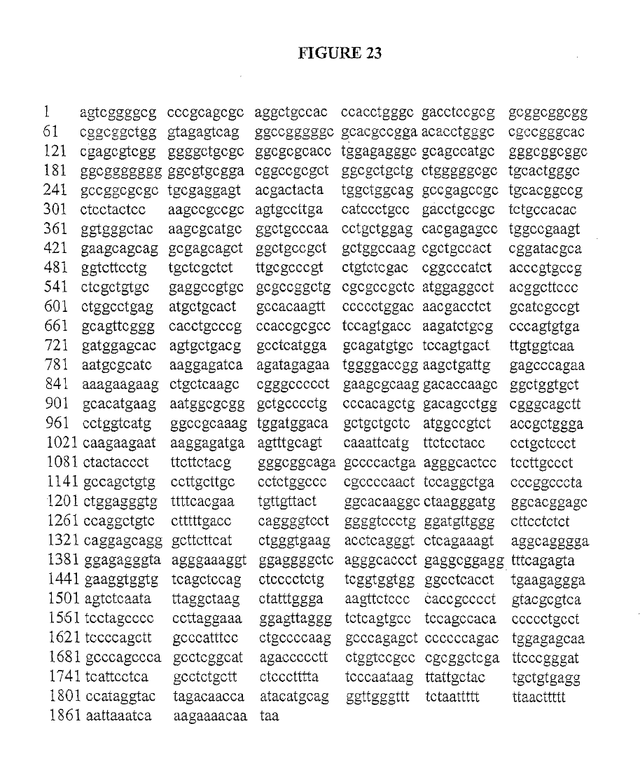

FIG. 24 shows the amino acid sequence for Wnt5a (SEQ ID NO: 4).

FIG. 25 shows adenovirus expressing sfrp-5c proteins. (A) Adenovirus expressing mouse sfrp5-Fc protein. Full-length mouse sfrp5 cDNA lacking the signal peptide (22-314AA) is obtained by polymerase chain reaction and subcloned into the EcoRI-BamHI site of Add2-Fc shuttle vector, which is a generous gift from Dr. Calvin Kuo. Secretion of Fc fusion protein into conditioned media is confirmed by transfection study. Add2-Fc shuttle vector is digested with PacI and co-transfected with pJM17 into 293 cells to allow for homologous recombination. Constructs are amplified in 293 cells and purified by ultracentrifugation in the presence of CsCl. (B) Adenovirus expressing human sfrp5-Fc protein.

### DETAILED DESCRIPTION

Sfrp5 is predicted to be a secreted protein based upon the presence of a signal peptide and the absence of a transmembrane domain. Sfrp5 is a member of the Sfrp family that contains a cysteine-rich domain homologous to the putative Wnt-binding site of Frizzled proteins. This family of protein acts as soluble modulators that sequesters Wnt proteins in the extracellular space between cells and prevents their binding to the receptors and antagonizes Wnt-mediated signaling pathways [6, 7], and canonical Wnt signaling negatively regulates adipogenesis [8]. However, to date, nothing is known about the role of Sfrp5 in regulation of obesity-associated metabolism on the control of non-canonical Wnt signaling in adipose tissue.

### A. Recombinant Human Sfrp5 and Fusion Proteins

Ideally, recombinant proteins are expressed as soluble proteins at high levels (i.e., greater than or equal to about 0.75% of total cellular protein, and more preferably, greater than 5% or even 15% of total cellular protein) in host cells. This facilitates the production and isolation of sufficient quantities in a highly purified form (i.e., substantially free of endotoxin or other pyrogen contamination).

In one embodiment, the present invention contemplates expressing and producing human Sfrp5 or fragment thereof as a fusion protein. In one embodiment, the fusion protein comprises a poly-histidine tract (also called a histidine tag). In one embodiment, the fusion protein comprises a portion of an antibody, e.g. the Fc fragment. The production of fusion proteins is not limited to the use of a particular expression vector and host strain. Several commercially available expression vectors and host strains can be used to express the C fragment protein sequences as a fusion protein containing a histidine tract. For example, Qiagen has a pQE xpression vector for mammalian cells.

### B. Detecting Markers In Vitro

In another embodiment, the present invention contemplates a method of measuring metabolic function (by measuring markers in tissue or blood, preferably plasma or serum), comprising: providing i) a sample (e.g. tissue, blood, secretion, etc.) from a subject and ii) a reagent (or other means) for measuring human Sfrp5 protein (or fragments thereof) or human Sfrp5 nucleic acid (or portions thereof); measuring the level of Sfrp5 protein or nucleic acid in the sample as an indicator of metabolic function.

In one embodiment, said reagent is an antibody reactive with human Sfrp5 protein. It is important to stress that commercially available antibodies that are advertised as reactive with human Sfrp5 protein were tested and found as not reactive or effective. Therefore, an antibody is contemplated against a unique human Sfrp5 sequence. In one embodiment, a polyclonal antibody against human Sfrp5 is generated by immunizing rabbits (or a monoclonal antibody is generated by immunizing mice or rats) with one or more synthetic peptides reflecting a unique portion of the human Sfrp sequence (such as WAPARCEEYDYYGWQAEP; SEQ ID NO: 5); in one embodiment, one or more peptides of this type are conjugated to KLH (e.g. through the Cys via maleimide linkage). In a preferred embodiment, said antibody is specific for human Sfrp5 (i.e. not reactive with other human proteins). In another embodiment, said antibody is reactive with human, but unreactive with mouse Sfrp5. The present invention contemplates these antibodies as compositions. In one embodiment, said reagent is an oligonucleotide probe (with a region of complementarity for human Sfrp5 nucleic acid) for measuring human Sfrp5 mRNA. In one embodiment, mRNA is measured in tissue biopsies.

### C. Treatment Approaches and Modalities

A variety of administration approaches (e.g. introducing an expression vector into a subject) and routes of administration may be used. However, it is preferred that administration of the recombinant protein (or fragment thereof) be done intravenously or through an implantable device that provides Sfrp5 in therapeutic quantities.

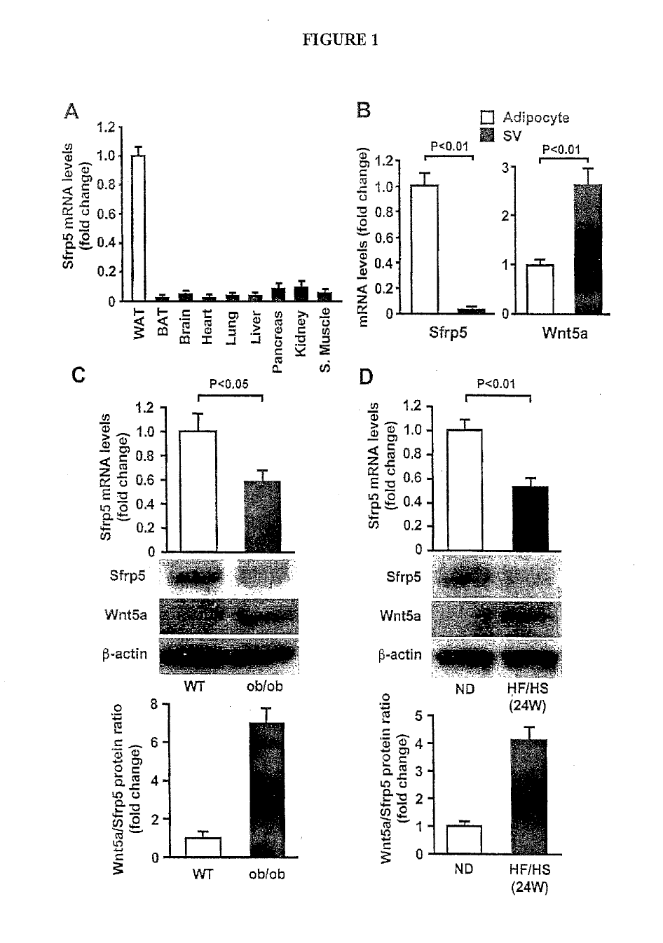

Because the dysregulation of adipokines can contribute to the pathophysiology of various obesity-linked disorders, we sought to identify new adipokine candidates by performing microarray analysis on the adipose tissues of lean and high-fat/high-sucrose (HF/HS) diet-induced obese mice [9]. The transcript encoding secreted frizzled-related protein (Sfrp) 5 was significantly higher in epididymal adipose tissue in obese mice fed HF/HS diet for 12 weeks than in lean mice fed a normal diet (FIG. 1A). Sfrp5 upregulation was transient, and expression declined to lower levels than that found in lean mice after 24 weeks of HF/HS diet feeding.

Sfrp5 is predicted to be a secreted protein based upon the presence of a signal peptide and the absence of a transmembrane domain using signal IP and SOUSI software, respectively. Sfrp5 is a member of the Sfrp family that contains a cysteine-rich domain homologous to the putative Wnt-binding site of Frizzled proteins. Without limiting the invention in any manner to any particular mechanism, it is believed that this family of protein acts as soluble modulators that sequesters Wnt proteins in the extracellular space between cells and prevents their binding to the receptors and antagonizes Wnt-mediated signaling pathways [6, 7], and canonical Writ signaling negatively regulates adipogenesis [8]. However, to date, nothing is known about the role of Sfrp5 in regulation of obesity-associated metabolism on the control of non-canonical Wnt signaling in adipose tissue.

Sfrp5 has been shown to bind and antagonize both Wnt5a and Wnt11 [10]. Furthermore, non-canonical Wnt pathway is activated by Wnt5a class ligands including Wnt5a and Wnt11 [11]. Thus, we assessed protein expression of Wnt5a and Wnt11 in epididymal fat tissues in WT mice fed normal or HF/HS diet by immunoblot analysis. Little or no expression of Wnt5a was observed in adipose tissues of normal diet-fed mice, but its level was increased in adipose tissue of HF/HS diet-fed obese mice after 12 and 24 weeks of HF/HS diet feeding (FIG. 1A). Of importance, HF/HS diet feeding for 12 weeks increased the Wnt5a/Sfrp5 protein ratio, and this ratio was further enhanced after 24 weeks of HF/HS diet feeding. In a similar manner to mice following 24 weeks of high caloric diet feeding, the genetic obese model, leptin-deficient ob/ob mice at the age of 20 weeks had an increase in Wnt5a expression and a decrease in Sfrp5 expression in epididymal fat tissue compared with WT mice, and Wnt5a/Sfrp5 ratio was higher in ob/ob mice than in WT mice (A). In contrast, no expression of Wmt11 protein was observed in fat of mice fed normal or HF/HS diet (data not shown).

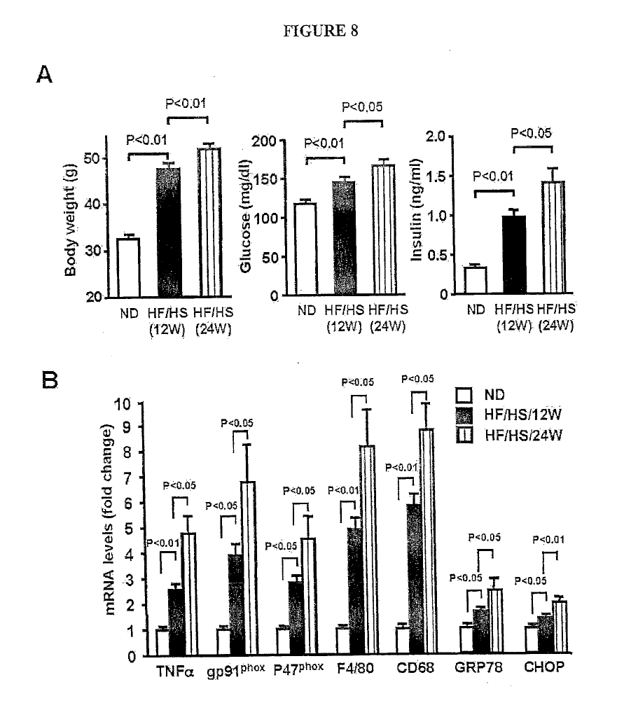

Because obesity-related metabolic dysfunction is attributed to inflammation and oxidative and ER stress in adipose tissue [12-16], the expression of TNFα, NADPH oxidase components gp91phox and P47phox, macrophage markers (F4/80 and CD68) and markers of ER stress (GRP78 and CHOP) was assessed in epididymal fat pads in lean mice and compared to that in obese mice fed HF/HS diet for 12 or 24 weeks. Corresponding to the increase in Wnt5a/Sfrp5 expression ratio, adipose expression of TNFα, gp91phox, P47phox, F4/80, CD68, GRP78 and CHOP was significantly higher in mice fed HF/HS diet for 12 weeks than in lean mice, and the levels of these transcripts were further elevated in mice fed HF/HS diet for 24 weeks (FIG. 8B). These findings are consistent with the observation showing that macrophage accumulation in fat tissue and glucose intolerance are exacerbated during the development of high caloric diet-induced obesity in mice [16].

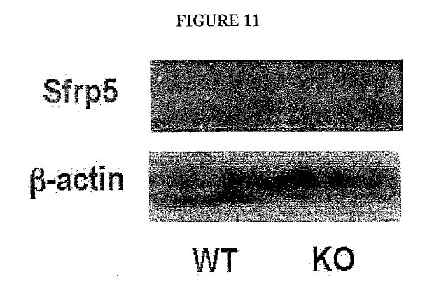

The visceral fat of obese individuals was scored for the presence of macrophage crown-like structures (CLS), an indicator of adipose tissue inflammation [17, 18]. CLS-positive individuals exhibited a decrease in Sfrp5 transcript expression compared with obese individuals that were negative for CLS (FIG. 11). CLS-positive individuals also displayed higher levels of adipose TNFα transcript expression and an increase in the insulin resistance marker HOMA-IR.

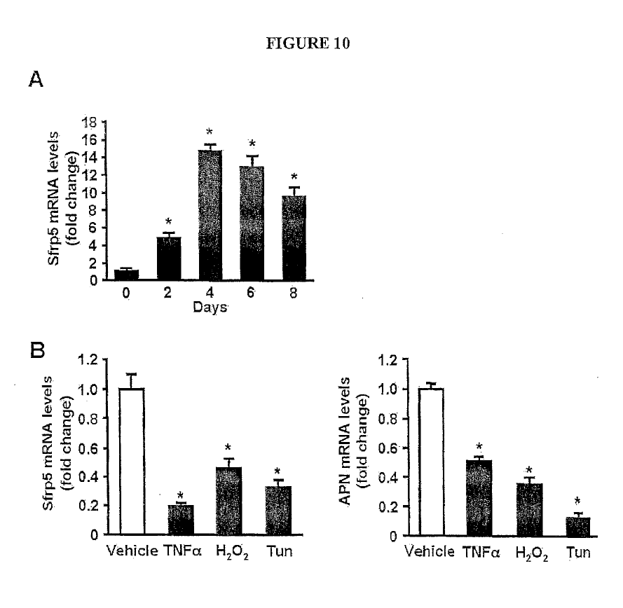

Sfrp5 expression by cultured 3T3-L1 adipocytes was up-regulated when cells were induced to differentiate, with the highest level of expression attained by day 4 (FIG. 1D). Sfrp5 expression was also assessed in 3T3-L1 adipocytes treated with agents to mimic various pathological states. Sfrp5 transcript levels were significantly reduced by treatment with TNFα or with inducers of oxidant and ER stress including hydrogen peroxide and tunicamycin, respectively (FIG. 1E). Similarly, the transcript level of adiponectin, a protective adipokine [2], was down-regulated in 3T3-L1 adipocytes by treatment with TNFα, hydrogen peroxide or tunicamycin (FIG. 10B). These data further document the dynamic regulation of Sfrp5 by adipocytes and they show that the inflammation and cellular stresses associated with obesity can account for the reduction of Sfrp5 expression in the fat tissues of mice that are either leptin-deficient or chronically fed a high-caloric diet, diabetic rats and obese subjects with insulin resistance.

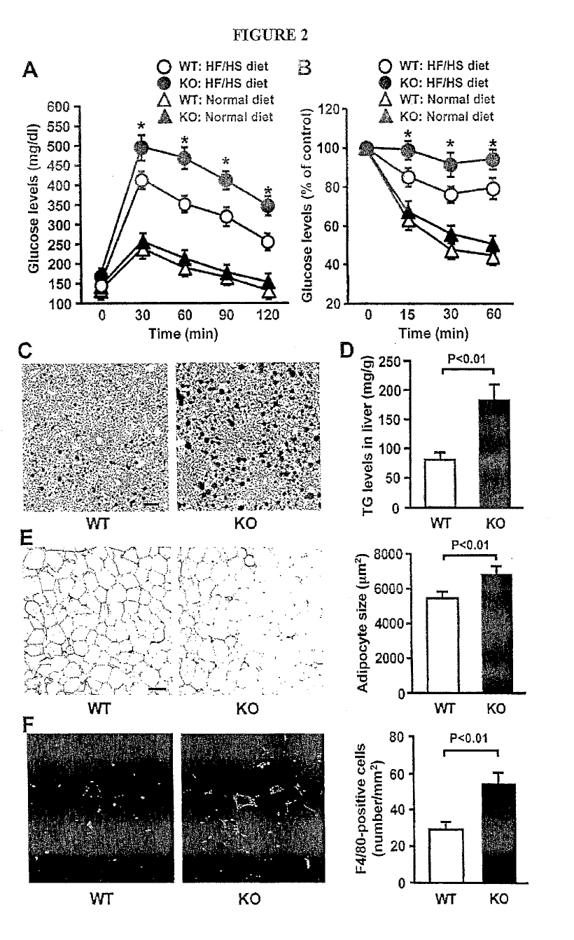

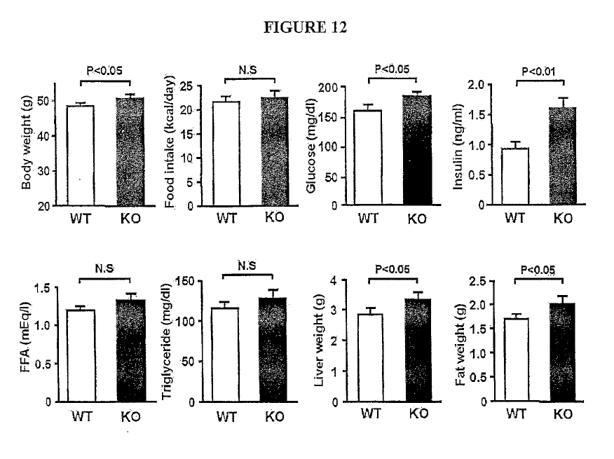

Sfrp5-deficient (Sfrp5^{-/-}) mice in a C57BL/6 background were used to investigate the pathophysiological role of Sfrp5 in the regulation of metabolism and adipose tissue inflammation. Sfrp5^{-/-} mice were fertile and viable. Despite the absence of Sfrp5 protein expression in adipose tissue (FIG. 11), no significant differences in BW (WT mice: 33.1±0.8 g and Sfrp5^{-/-} mice: 34.1±1.1 g), glucose disposal, or insulin sensitivity could be detected between Sfrp5^{-/-} and wild-type (WT) mice when mice were fed a standard chow diet (FIGS. 2A and B). However, after HF/HS diet feeding for 12 weeks, Sfrp5^{-/-} mice showed a significant impairment in glucose clearance and insulin sensitivity compared to WT mice (FIGS. 2A and B). Although Sfrp5^{-/-} mice showed a small but significant increase in BW compared with WT mice (FIG. 12), both strains had similar daily food intake during the experimental period of HF/HS diet feeding (FIG. 12). Fasting glucose and insulin levels in serum were elevated in Sfrp5^{-/-} mice compared with WT mice, but serum levels of free fatty acids and triglyceride did not significantly differ between two strains (FIG. 12). Immunohistochemical analysis of liver stained with oil red O revealed a greater degree of hepatic steatosis, with a higher triglyceride content and heavier liver weight, in Sfrp5^{-/-} mice as compared with WT mice on the HF/HS diet ((FIGS. 2C and D, and F). Histological analyses were performed on epididymal adipose tissues. Sfrp5^{-/-} mice fed a HF/HS diet had adipocytes with larger cross-section area than HF/HS diet-fed WT mice (FIG. 2D and FIG. 12). These results are consistent with the observation that epididymal fat tissue mass was greater in Sfrp5^{-/-} mice than in WT mice after HF/HS feeding (FIG. 12). Collectively, these data show that Sfrp5-deficiency does not lead to an observable phenotype under normal nutritional conditions; however, under conditions of metabolic stress, Sfrp5-deficiency leads to a higher degree of metabolic dysfunction.

Histological analyses were performed on epididymal adipose tissues. Sfrp5^{-/-} mice fed a HF/HS diet had adipocytes with larger cross-section area than HF/HS diet fed WT mice (FIG. 2F). These results are consistent with the observations showing that the weight of epididymal fat tissues was slightly but statistically significantly heavier in Sfrp5^{-/-} mice than in WT mice after HF/HS feeding (Fat weight: 1.64±0.08 g in WT mice and 1.94±0.15 g in Sfrp5^{-/-} mice, p<0.05).

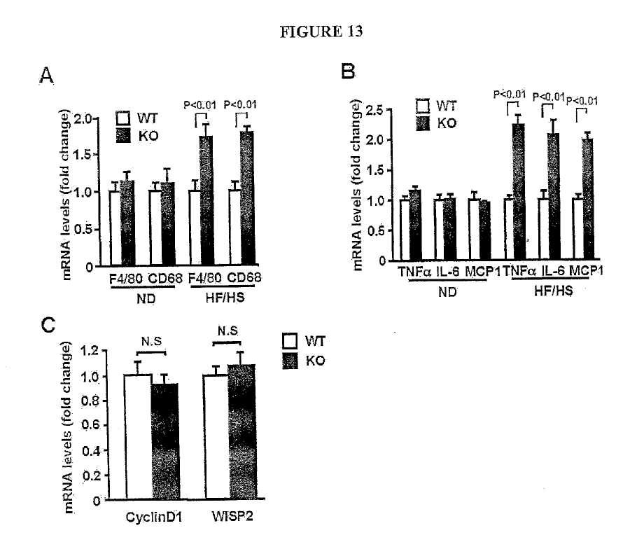

An increased inflammatory response in adipose tissues is linked to the development of insulin resistance and glucose intolerance [14-16]. Thus, macrophage content in the adipose tissue of Sfrp5^{-/-} and WT mice was assessed. Cells positive for F4/80, a macrophage marker, appeared at a significantly higher frequency in adipose tissue of Sfrp5^{-/-} mice compared to WT mice when both strains were fed a HF/HS diet ((FIG. 2F). Consistent with this finding, transcript levels of F4/80 and CD68 were significantly elevated in epididymal adipose tissue of Sfrp5^{-/-} mice compared with WT mice (FIG. 13A). In contrast, F4/80 and CD68 expression levels were not different between two groups of mice when fed a normal diet. The expression levels of pro-inflammatory cytokines and a chemokine was determined in the stromal vascular fractions isolated from epididymal adipose tissue to assess the status of macrophage activation. When fed HF/HS diet, significant increases in levels of TNFα, IL-6 and MCP-1 transcripts occurred in the stromal vascular fraction from fat tissue of Sfrp5^{-/-} mice compared with WT mice (FIG. 13B). Transcript levels of TNFα, IL-6 and MCP-1 did not differ between Sfrp5^{-/-} and WT mice when fed a normal chow diet.

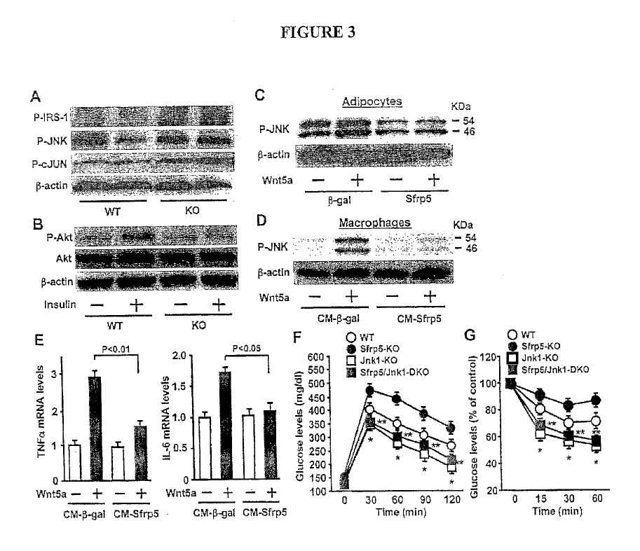

The pathways involved in canonical or non-canonical Wnt signaling were assessed in epididymal adipose tissues of Sfrp5^{-/-} and WT mice fed a HF/HS diet. No differences were detected in transcript expression of cyclin D1 or WISP2, indicators of canonical Wnt signaling, between Sfrp5^{-/-} and WT mice (FIG. 13C). In contrast, the phosphorylation of c-Jun N terminal kinase (JNK), a downstream target of the non-canonical Wnt signaling [11, 19], was elevated 2.0±0.1 (P<0.05) in white adipose tissue in Sfrp5^{-/-} mice, and the phosphorylation of c-Jun, a downstream substrate of JNK, was elevated in HF/HS diet-fed Sfrp5^{-/-} mice by a factor of 2.3±0.2 (P<0.05) compared with WT mice on a HF/HS diet (FIG. 3A). Activation of JNK1 is reported to promote insulin resistance through serine phosphorylation of IRS-1 at specific residues [20]. Correspondingly, MS-1 phosphorylation at residue Ser307 was increased in fat tissue of Sfrp5^{-/-} mice by a factor of 2.2±0.3 (P<0.05) compared with WT mice (FIG. 3A). Insulin signaling in adipose tissue was also assessed by measuring the activating phosphorylation of Akt at Ser473 following insulin administration. In WT mice fed a HF/HS diet, insulin stimulated the phosphorylation of Akt in fat pads, but this induction was diminished in the HF/HS diet-fed Sfrp5^{-/-} mice (FIG. 3B). Because activation of JNK causes obesity-induced insulin resistance and glucose intolerance [20], we hypothesized that the severe metabolic dysfunction observed in HF/HS diet-fed Sfrp5^{-/-} mice could be attributed to the non-canonical activation of JNK in fat tissues.

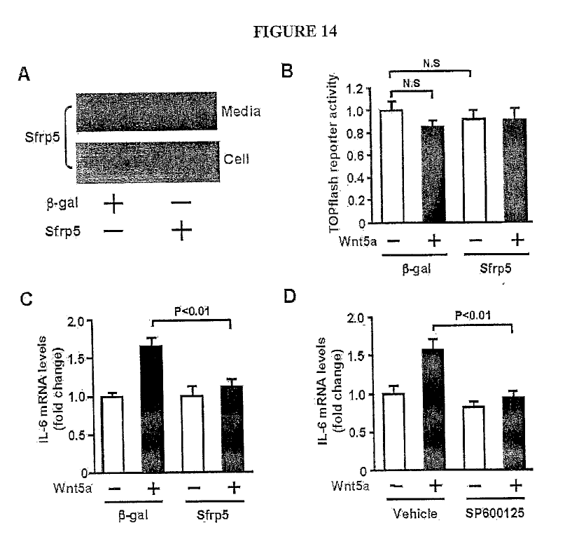

To assess the effect of Sfrp5 and Wnt5a on JNK activation at the cellular level, 3T3-L1 adipocytes were transduced with adenoviral vectors expressing Sfrp5 (Ad-Sfrp5) or β-galactosidase (Ad-β-gal) as control, followed by incubation with Wnt5a protein. Transduction of 3T3-L1 cells with Ad-Sfrp5 led to an increase in Sfrp5 protein in both cell lysates and media compared with Ad-β-gal (FIG. 14A). Moreover, transduction with Ad-Sfrp5 cancelled the stimulatory effects of Wnt5a on phosphorylation of JNK in adipocytes (FIG. 3C). Consistent with findings from mice, neither Sfrp5 nor Wnt5a had an effect on TOPflash reporter activity, which contains TCF binding sites and responds to canonical Wnt signaling (FIG. 14B).

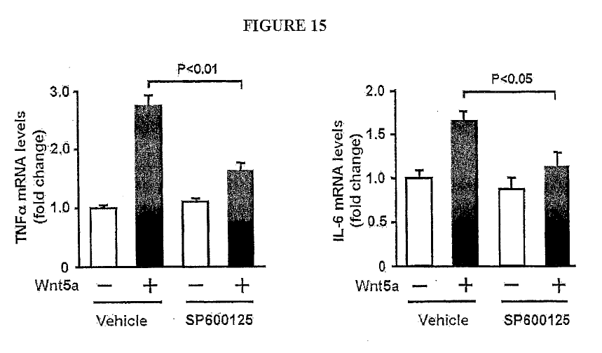

To assess the effect of Sfrp5 on JNK activation and inflammatory response in macrophages in vitro, cultured murine macrophages were stimulated with Wnt5a protein in the presence of conditioned media from 3T3-L1 adipocytes transduced with Ad-Sfrp5 or Ad-β-gal. Wnt5a-treatment stimulated JNK phosphorylation in macrophages in conditioned media from Ad-β-gal-treated 3T3-L1 adipocytes, which was blocked by the conditioned media from Ad-Sfrp5-transduced adipocytes (FIG. 3D). Treatment with Wnt5a also increased TNFα and IL-6 transcript expression by macrophages and this was blocked by conditioned media from Ad-Sfrp5-transduced adipocytes (FIG. 3E). To test the contribution of JNK signaling to Wnt5a-stimulated induction of TNFα and IL-6, macrophages were pretreated with the JNK inhibitor SP600125 and incubated with Wnt5a. Pretreatment with SP600125 diminished Wnt5a-stimulated expression of TNFα and IL-6 (FIG. 15), indicating that Sfrp5 blocks macrophage activation through inhibition of Wnt5a-JNK signaling. Similarly, the stimulatory effects of Wnt5a on IL-6 expression in adipocytes were blocked by transduction with Ad-Sfrp5 or pretreatment with SP600125 (FIGS. 14C and D).

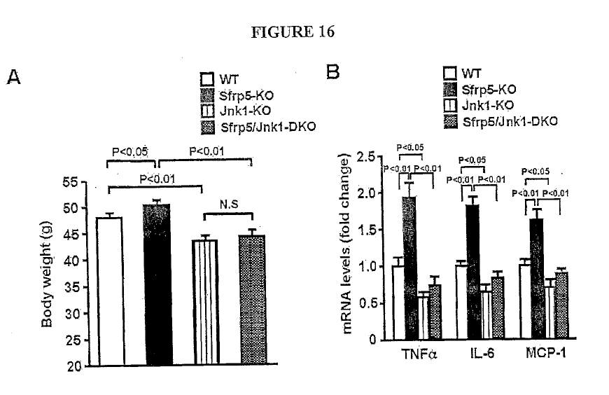

Mice lacking both Sfrp5 and Jnk1 were generated to investigate the causal role of JNK1 activation in the severe diet-induced metabolic dysfunction and adipose tissue inflammation that develops in the Sfrp5-deficient mice. Consistent with a previous report [20], Jnk1^{-/-} mice exhibited improvements in insulin resistance and glucose intolerance, and reduced BW compared with WT mice when fed the HF/HS diet (FIGS. 3F and G, and FIG. 16A). Whereas Sfrp5^{-/-} mice showed profound insulin-resistance and glucose intolerance, and a small increase in BW, Sfrp5^{-/-}Jnk1^{-/-} double-knockout mice showed glucose disposal responses in glucose tolerance and insulin tolerance tests and BW that were comparable with Jnck^{-/-} mice (FIGS. 3F and G, and FIG. 16A). Furthermore, while transcript levels of TNFα, IL-6 and MCP-1 in fat tissue were elevated in Sfrp5^{-/-} mice compared to WT mice, their expression levels did not differ between Jnk1^{-/-} and Sfrp5^{-/-}Jnk1^{-/-} mice (FIG. 16B). Thus, the impaired insulin sensitivity and enhanced adipose tissue inflammation in Sfrp5^{-/-} mice can be attributed to enhanced activation of JNK1.

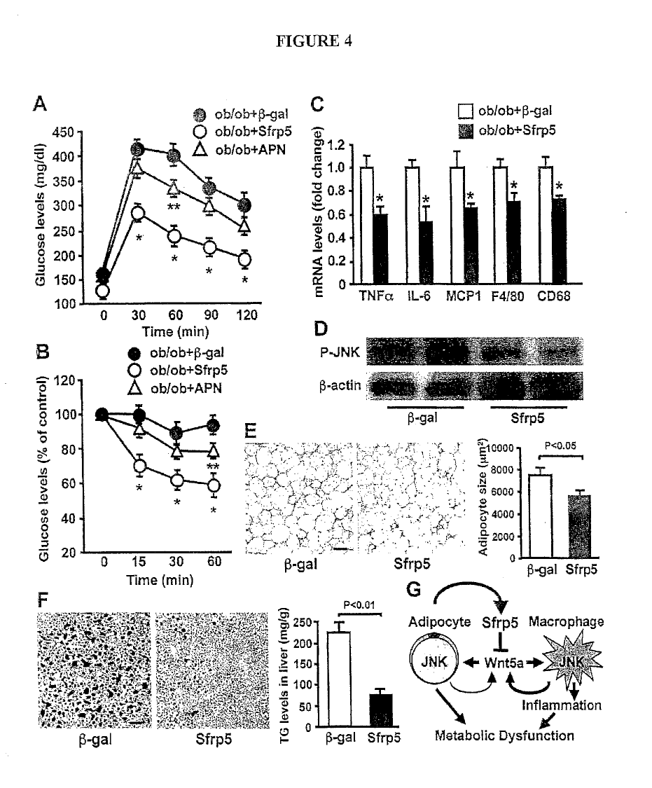

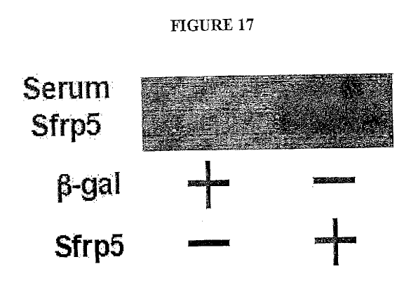

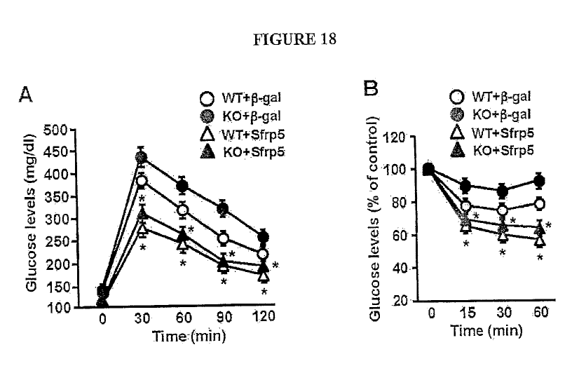

To test whether the over-expression of Sfrp5 is protective against the development of insulin resistance and glucose intolerance in vivo, we intravenously administered Ad-Sfrp5 or Ad-β-gal to WT and Sfrp5^{-/-} mice that were fed a HF/HS diet for 10 weeks. Detectable circulating levels of Sfrp5 could be measured in serum one week after the delivery of Ad-Sfrp5 (FIG. 17). Both WT and Sfrp5^{-/-} mice treated with Ad-Sfrp5 exhibited significant improvements in glucose clearance in glucose and insulin tolerance tests compared with mice treated with the control vector (FIGS. 18A and B). To investigate the effect of acute Sfrp5 delivery on glucose metabolism in another mouse model of metabolic dysfunction, we systemically injected Ad-Sfrp5 or Ad-β-gal into ob/ob mice at the age of 20 weeks. Parallel experiments examined the consequences of intravenous injection of adenoviral vectors expressing APN (Ad-APN) because the chronic overexpression of this adipokine has been shown to reverse the metabolic consequences of leptin deficiency [21]. Two weeks after treatment with Ad-Sfrp5, glucose clearance was significantly improved as assessed by glucose tolerance (FIG. 4A) and insulin tolerance assays (FIG. 4B). Administration of Ad-APN to ob/ob mice led to a 3-fold increase in plasma adiponectin levels at 7 days after injection. However, the acute administration of this adipokine was relatively ineffective in improving glucose clearance in this model (FIGS. 4A and B). The administration of Ad-Sfrp5 led to significant reductions in transcript levels of TNFα, IL-6, MCP-1, F4/80 and CD68 in the adipose tissue of ob/ob mice (FIG. 4C). The Sfrp5-mediated improvements in glucose metabolism and inflammatory marker expression in ob/ob mice were accompanied by a reduction in the activating phosphorylation of JNK in adipose tissue (FIG. 4D). Treatment with Ad-Sfrp5 also led to the atrophy of enlarged white adipocytes in ob/ob mice (FIG. 4E) with a reduction of fat weight (Ad-β-gal: 2.51±0.19 g and As-Sfrp5: 2.01±0.11 g, p<0.05). Furthermore, treatment of ob/ob mice with Ad-Sfrp5 resulted in a marked attenuation of lipid accumulation in the liver (FIG. 4F). Taken together, these data indicate that acute Sfrp5 administration can reverse hyperglycemia and hepatic steatosis in multiple mouse models of metabolic dysfunction.

The development of obesity-related metabolic disorders is attributed in part to an imbalance in the production of adipokines, most of which are pro-inflammatory and detrimental to metabolism. In comparison, adiponectin is unique among adipokines in that it has anti-inflammatory and insulin-sensitizing actions in a number of models. Here we show that Sfrp5 is secreted by adipocytes and that it regulates the microenvironment of white adipose tissue by regulating macrophage activation under conditions of metabolic stress. Whereas Sfrp5^{-/-} mice do not express a detectable phenotype when fed a normal chow diet, these animals displayed aggravated fat pad inflammation and systemic metabolic dysfunction when fed a high calorie diet. Conversely, the acute administration of Sfrp5 to models of obese and diabetic mice improved metabolic function and reduced adipose tissue inflammation. Notably, the salutary actions of Sfrp5 administration on glucose metabolism were more effective when compared with the acute over-production of adiponectin in ob/ob mice.

Our data indicate that Sfrp5 can function to neutralize noncanonical JNK activation by Wnt5a in macrophages and adipocytes via paracrine and autocrine mechanisms, respectively (FIG. 4G). The activation of JNK signaling in adipocytes and macrophages has emerged as an important mediator of adipose tissue inflammation that affects systemic metabolism [20, 22-24]. Thus, the Sfrp5-JNK1 regulatory axis in fat represents a new target for the control of obesity-linked glucose homeostasis.

### D. Antibodies to Wnt5a

As noted above, one approach to treatment involves administering human Sfrp5 protein to a subject. However, because of the findings described above, another approach to treatment is contemplated, i.e. administering antibodies to Wnt5a. Thus, in one embodiment, the present invention contemplates a method of reducing elevated glucose levels, comprising: a) providing a subject with elevated glucose levels and a composition comprising an antibody or portion thereof reactive with human Wnt5a; b) administering said composition to said subject; and measuring said glucose levels of said subject until they are reduced. It is preferred that said subject is a human and said antibody is a humanized monoclonal antibody reactive with human Wnt5a.

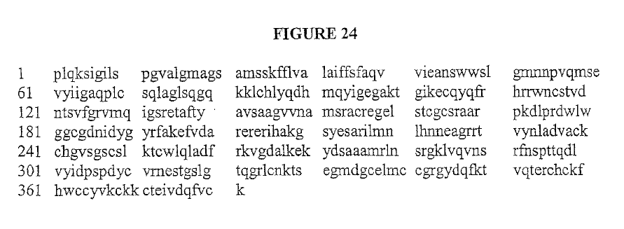

It is not intended that the present invention be limited to a particular antibody of method of making an anti-Wnt5a antibody. FIG. 24 shows the amino acid sequence for Wnt5a. In one embodiment, the entire protein is used for immunization. In another embodiment, a peptide portion (e.g. PKDLPRDWLW SEQ ID NO: 6) is used for immunization. In one embodiment, such a peptide is administered with an adjuvant, e.g. KLH.

### E. Implantable Devices

In one embodiment, the present invention contemplates the use of an implant or implantable device to provide either human Sfrp5 protein or portion thereof (directly as a protein or via an expression vector, including a vector in host cells) or antibody to Wnt5a. There are a variety of devices available and it is not intended that the present invention be limited to a particular device. For example, there are devices comprising tubular matrices (U.S. Pat. No. 6,716,225, hereby incorporated by reference [25]) which can be applied in this manner. In one embodiment, the implant comprises polymeric gel material containing bioactive molecules (see U.S. Pat. No. 6,290,729, hereby incorporated by reference [26]). In one embodiment, the implant comprises a sponge-like structure having a plurality of convoluted capillaries and from which the active material is released (see U.S. Pat. No. 4,587,267, hereby incorporated by reference [27]).

### EXPERIMENTAL

### Materials and Methods

Phospho-Akt (Ser473), phospho-JNK (Thr183/Tyr185), phospho-cJUN (Ser63), Akt, and GAPDH antibodies were purchased from Cell Signaling Technology. Phospho-IRS-1 (Ser307) antibody was purchased from Upstate biotechnology. Tunicamycin, H₂O₂ and β-actin antibody were purchased from Sigma Chemical Co. The polyclonal antibody against mouse Sfrp5 was generated by immunizing rabbits with two synthetic peptides conjugated to KLH through the Cys via maleimide (APTRGQEYDYYGWQAEP: amino acid residues 22-38 (SEQ ID NO: 7) and Acetyl-VKMRIKEIKIDNGDRKLIG: amino acid residues 201-219) (21st Century Biochemicals) (SEQ ID NO: 8). SP600125 was purchased from Biomol international. Recombinant mouse Wnt5a protein produced in CHO cells (endotoxin free: <1.0 EU/μg protein by the LAL method), mouse Wnt5a antibody and mouse TNFα proteins were purchased from R&D Systems.

### Mouse Model

Mice lacking Sfrp5 were backcrossed and maintained on the C57BL6/J background. Sfrp5^{-/-} mice were generated by replacing the first protein coding exon with the PGKneobpAloxA cassette as described previously [28]. Sfrp5^{-/-} mice and littermate wild-type (WT) C57BL6/J mice were used. To generate mice lacking both Sfrp5 and JNK1, Sfrp5^{-/-} mice and Jnk1^{-/+} mice (Jackson laboratory) were inbred. Ob/ob mice were purchased from Jackson laboratory. Study protocols were approved by the Boston University Institutional Animal Care and Use Committee. Mice were fed either a normal chow diet (Harlan Teklad global 18% protein rodent diet, #2018) or a HF/HS diet (Bio-Serv, #F1850) [9] as indicated. The composition of the HF/HS diet was 35.8% fat (primarily lard), 36.8% carbohydrate (primarily sucrose), and 20.3% protein. For the high caloric diet feeding, mice at the age of 10 weeks were maintained on a HF/HS diet for 12 or 24 weeks.

### Collection of Human Visceral Fat Tissue

Visceral adipose tissue was collected during gastric bypass surgery in obese human adults (age ≥21 years) with a body mass index ≥30 kg/m² as described previously [17]. Patients with unstable medical conditions such as active coronary syndromes, congestive heart failure, systemic infection, malignancy, or pregnancy were excluded. The presence or absence of macrophage crown-like structures (CLS) in adipose tissue was determined by immunohistochemical stains with CD68 in a blinded manner as described previously [17]. The insulin resistance marker, homeostasis model assessment of insulin resistance (HOMA-IR) were quantified from blood samples [17]. All subjects gave written, informed consent, and the study was approved by the Boston University Medical Center Institutional Review Board.

### Cell Culture

Mouse 3T3-L1 cells (ATCC) were maintained in DMEM with 10% fetal bovine serum (FBS) and differentiated into adipocytes by treatment with DMEM supplemented with 5 μg/ml of insulin, 0.5 mM 1-methyl-3-isobutyl-xanthin, and 1 μM dexamethazone [29]. At day 7 after differentiation, 3T3-L1 adipocytes were treated with tunicamycin, H₂O₂, TNFα or vehicle for 24 h. Peritoneal macrophages from lean WT C57BL/6 mice were maintained in DMEM supplemented 10% FBS and placed in DMEM with 0.5% FBS for 16 h for serum starvation. Macrophages were incubated in the conditioned media from 3T3-L1 adipocytes transduced with adenoviral vectors in the presence of Wnt5a protein (200 ng/ml) or vehicle for 24 h. In some experiments, cells were pretreated with SP600125 (15 μM) or vehicle for 1 h followed by treatment with Wnt5a protein.

Construction of Adenoviral Vectors

For transduction experiments, tetracycline-regulated adenovirus (Ad) vectors were used [30]. The Ad vectors expressing β-galactosidase (β-gal) or Sfrp5 were constructed under the control of seven consecutive tetracycline-responsive elements (TRE) and a CMV minimal promoter (AdTRE-β-gal or AdTRE-Sfrp5) Co-transfection of AdTRE vector with Ad vectors encoding tetracycline transactivator (tTA, a fusion of TRE-binding protein and VP16 transactivation domain) under the control of the CMV promoter/enhancer (AdCMV-tTA) results in activation of transgene. Ad vectors expressing adiponectin (Ad-APN) under control of the CMV promoter was constructed as previously described [31, 32]. For in vivo gene transfer, AdTRE-β-gal or AdTRE-Sfrp5 along with AdCMV-tTA (2.5×10⁸ pfu for each adenovirus) was injected into the jugular vein of mice. For some experiments, Ad-APN (5.0×10⁸ pfu total) was intravenously administered to mice. For in vitro transduction, cells were transfected with AdTRE-β-gal or AdTRE-Sfrp5 in the presence of AdCMV-tTA (125 MOI for each adenovirus) for 24 h. The media was then replaced with fresh DMEM, and cells were incubated for additional 24 h. Cells were treated with or without recombinant Wnt5a protein (200 ng/ml) for indicated length of time. For detection of Sfrp5 in media, protein was concentrated 3-fold with a Microcon column.

### Luciferase Reporter Assays

3T3-L1 adipocytes were transduced with adenoviral vectors for 24 h and co-transfected with a TOPflash (Upstate) construct and a Renilla luciferase control plasmid (pRL-SV40, Promega) to normalize for transfection efficiency. After transfection, cells were treated with Wnt5a protein (200 ng/ml) or vehicle. Cells were lysed and analyzed on a luminometer by using dual luciferase assay kit (Promega).

### Metabolic Measurements

Serum insulin levels were determined by ELISA using mouse insulin as a standard (Crystal Chemical Inc.). Serum glucose, free fatty acid and triglyceride levels were measured with enzymatic kits (Wako Chemicals). Glucose tolerance testing was performed on 6 hr-fasted mice injected intraperitoneally with D-glucose (1 g/kg body weight) [9]. Blood glucose levels were determined immediately before, and 30, 60, 90, and 120 min after injection as determined by an Accu-Chek glucose monitor (Roche Diagnostics Corp.). Insulin tolerance testing was performed on 6 hr-fasted mice injected intraperitoneally with human insulin (Humulin R, Eli Lilly) at 1.5 U/kg body weight for lean mice or at 4.5 U/kg body weight for obese mice. Blood glucose levels were determined immediately before and 15, 30, and 60 min after injection. Insulin signaling in adipose tissues was determined by measurement of Akt phosphorylation following insulin administration. Mice were fasted overnight and treated with insulin (4.5 U/kg body weight) or saline via the inferior vena cava. Epididymal fat was isolated after 4 min, and immunoblot analysis was preformed. To determine triglyceride content of liver, lipid was extracted using the Bligh-Dyer method [33]. Liver tissues were homogenized in chloroform/MeOH/H₂O (1:2:0.8) and centrifuged. Supernatants were collected, and equal amounts of chloroform and H₂O were added. After vortexing and centrifugation, the chloroform layer was collected, dried completely and resuspended in isopropanol containing 10% Triton-X. Triglyceride levels were measured with enzymatic kits (Wako Chemicals).

### Histology

The sections of epididymal adipose tissue were fixed in 10% formalin, dehydrated, and embedded in paraffin. Adipose tissue sections were stained with hematoxylin and eosin to examine the morphology and with anti-F4/80 antibody (Santa Cruz) to detect macrophages. Adipocyte cross-sectional areas were measured in 200 cells per mouse using Image J software. Macrophage accumulation was quantified by measuring the number of F4/80-positive cells per mm² in 20 randomly chosen microscopic fields. Liver tissues were embedded in OCT compound (Sakura Finetech USA Inc.) and snap frozen in liquid nitrogen and stained with oil red O for lipid deposition by standard methods [9].

### Isolation of Mouse Adipocyte and Stromal Vascular (SV) Fraction

Epididymal fat pads from male wild-type C57BL/6 mice fed a normal diet were excised, minced in PBS and digested with 1 mg/ml collagenase Type 1 (Worthington Chemical Corporation) at 37° C. for 30 min. The digested fat tissue was filtered through a mesh and centrifuged at 1000 rpm for 5 min to separate floating adipocytes from the SV fraction.
Measurement of mRNA Levels
Gene expression level was quantified by real-time PCR. Total RNA was prepared with the use of a Qiagen kit. cDNA was produced using ThermoScript RT-PCR Systems (Invitrogen). PCR was performed on iCycler iQ Real-Time PCR Detection System (Bio-Rad) using SYBR Green 1 as a double standard DNA specific dye (Applied Biosystems).

Primers were:
(SEQ ID NO: 9)
5′-GAAAGTTGATTGGAGCCCAGAA-3′
and
(SEQ ID NO: 10)
5′-GCCCGTCAGGTTGTCTAACTGT-3′ for mouse Sfrp5,
(SEQ ID NO: 11)
5′-CAGCCAGATGCAGTTAACGC-3′
and
(SEQ ID NO: 12)
5′-GCCTACTCATTGGGATCATCTTG-3′ for mouse MCP-1,
(SEQ ID NO: 13)
5′-CTTTGGCTATGGGCTTCCAGTC-3′
and
(SEQ ID NO: 14)
5′-GCAAGGAGGACAGAGTTTATCGTG-3′ for mouse F4/80,
(SEQ ID NO: 15)
5′-CTTCTGCTGTGGAAATGCAA-3′
and
(SEQ ID NO: 16)
5′-AGAGGGGCTGGTAGGTTGAT-3′ for mouse CD68,
(SEQ ID NO: 17)
5′-TGCCATCCATGCGGAAA-3′
and
(SEQ ID NO: 18)
5′-AGCGGGAAGAACTCCTCTTC-3′ for mouse cyclinD1,
(SEQ ID NO: 19)
5′-ATACAGGTGCCAGGAAGGTG-3′
and
(SEQ ID NO: 20)
5′-CAAGGGCAGAAAGTTGGTGT-3′ for mouse WISP2,
(SEQ ID NO: 21)
5′-AGGTTGGATGGCAGGC-3′
and
(SEQ ID NO: 22)
5′-GTCTCACCCTTAGGACCAAGAA-3′ for mouse adiponectin,
(SEQ ID NO: 23)
5′-TTGGGTCAGCACTGGCTCTG-3′
and
(SEQ ID NO: 24)
5′-TGGCGGTGTGCAGTGCTATC-3′ for mouse gp91^{phox},
(SEQ ID NO: 25)
5′-GATGTTCCCCATTGAGGCCG-3′
and
(SEQ ID NO: 26)
5′-GTTTCAGGTCATCAGGCCGC-3′ for mouse P47^{phox},
(SEQ ID NO: 27)
5′-ACCTATTCCTGCGTCGGTGT-3′
and
(SEQ ID NO: 28)
5′-GCATCGAAGACCGTGTICTC-3′ for mouse GRP78,
(SEQ ID NO: 29)
5′-GTCCTGTCCTCAGATGAAATTGG-3′
and
(SEQ ID NO: 30)
5′-GCAGGGTCAAGAGTAGTGAAGGTT-3′ for mouse CHOP,
(SEQ ID NO: 31)
5′-AATCAAGAACGAAAGTCGGAGG-3′
and
(SEQ ID NO: 32)
5′-GCGGGTCATGGGAATAACG-3′ for 18S,
and
(SEQ ID NO: 33)
5′-GCTCCAAGCAGATGCAGCA-3′
and
(SEQ ID NO: 34)
5′-CCGGATGTGAGGCAGCAG-3′ for mouse 36B4.
Primers for mouse TNFα and mouse IL-6 were
purchased from Qiagen.

### Western Blot Analysis

Tissue and cell samples were homogenized, and equal amounts of proteins were separated with denaturing SDS 4-15% polyacrylamide gels. Following transfer to membranes, immunoblot analysis was performed with the indicated antibodies followed by incubation with secondary antibody conjugated with horseradish peroxidase at a 1:5000 dilution. ECL Western Blotting Detection kit (Amersham Pharmacia Biotech) or ECL Advance Western Blotting Detection kit (Amersham Pharmacia Biotech) was used for detection. Relative phosphorylation or protein levels were quantified by Image J program.

### Statistical Analysis

All data are expressed as means±SEM. Differences were analyzed by Student's unpaired t test or analysis of variance (ANOVA) for multiple comparisons. A value of P<0.05 was accepted as statistically significant.

### EXAMPLE 1

### Construction and Expression of Fusion Proteins

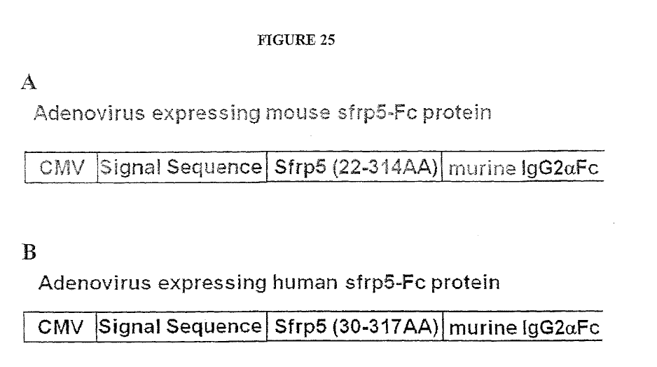

FIG. 25 provides a design strategy for fusion proteins of both mouse and human Sfrp5. In both instances, the fusion partner is a portion of an immunoglobulin. In one embodiment, the present invention contemplates that the fusion partner for the mouse construct is a portion of a mouse immunoglobulin. In another embodiment, the fusion partner for the human construct is a portion of a human immunoglobulin. For example, U.S. Pat. No. 7,670,595 (hereby incorporated by reference [34]) describes a number of human Fc regions useful for producing fusion proteins.

### REFERENCES

1. Spiegelman, B. M., and Flier, J. S. (1996) Adipogenesis and obesity: rounding out the big picture, Cell 87, 377-389.
2. Ouchi, N., Kihara, S., Funahashi, T., Matsuzawa, Y., and Walsh, K. (2003) Obesity, adiponectin and vascular inflammatory disease, Curr Opin Lipidol 14, 561-566.
3. Scherer, P. E. (2006) Adipose tissue: from lipid storage compartment to endocrine organ, Diabetes 55, 1537-1545.
4. Ouchi, N., Shibata, R., and Walsh, K. (2006) Cardioprotection by adiponectin, Trends Cardiovasc Med 16, 141-146.
5. Winter, G. P. “Recombinant altered antibodies and methods of making altered antibodies,” U.S. Pat. No. 5,225,539 (published Jul. 6, 1993).
6. Bovolenta, P., Esteve, P., Ruiz, J. M., Cisneros, E., and Lopez-Rios, J. (2008) Beyond Wnt inhibition: new functions of secreted Frizzled-related proteins in development and disease, J Cell Sci 121, 737-746.
7. Kawano, Y., and Kypta, R. (2003) Secreted antagonists of the Wnt signalling pathway, J Cell Sci 116, 2627-2634.
8. Ross, S. E., Hemati, N., Longo, K. A., Bennett, C. N., Lucas, P. C., Erickson, R. L., and MacDougald, O. A. (2000) Inhibition of adipogenesis by Wnt signaling, Science 289, 950-953.
9. Izumiya, Y., Hopkins, T., Morris, C., Sato, K., Zeng, L., Viereck, J., Hamilton, J. A., Ouchi, N., LeBrasseur, N. K., and Walsh, K. (2008) Fast/Glycolytic muscle fiber growth reduces fat mass and improves metabolic parameters in obese mice, Cell Metab 7, 159-172.
10. Li, Y., Rankin, S. A., Sinner, D., Kenny, A. P., Krieg, P. A., and Zorn, A. M. (2008) Sfrp5 coordinates foregut specification and morphogenesis by antagonizing both canonical and noncanonical Wnt11 signaling, Genes Dev 22, 3050-3063.
11. Veeman, M. T., Axelrod, J. D., and Moon, R. T. (2003) A second canon. Functions and mechanisms of beta-catenin-independent Wnt signaling, Dev Cell 5, 367-377.
12. Furukawa, S., Fujita, T., Shimabukuro, M., Iwaki, M., Yamada, Y., Nakajima, Y., Nakayama, O., Makishima, M., Matsuda, M., and Shimomura, I. (2004) Increased oxidative stress in obesity and its impact on metabolic syndrome, J Clin Invest 114, 1752-1761.
13. Gregor, M. G., and Hotamisligil, G. S. (2007) Adipocyte stress: The endoplasmic reticulum and metabolic disease, J Lipid Res.
14. Hotamisligil, G. S. (2006) Inflammation and metabolic disorders, Nature 444, 860-867.
15. Weisberg, S. P., McCann, D., Desai, M., Rosenbaum, M., Leibel, R. L., and Ferrante, A. W., Jr. (2003) Obesity is associated with macrophage accumulation in adipose tissue, J. Clin. Invest. 112, 1796-1808.
16. Xu, H., Barnes, G. T., Yang, Q., Tan, G., Yang, D., Chou, C. J., Sole, J., Nichols, A., Ross, J. S., Tartaglia, L. A., and Chen, H. (2003) Chronic inflammation in fat plays a crucial role in the development of obesity-related insulin resistance, J Clin Invest 112, 1821-1830.
17. Apovian, C. M., Bigornia, S., Mott, M., Meyers, M. R., Ulloor, J., Gagua, M., McDonnell, M., Hess, D., Joseph, L., and Gokce, N. (2008) Adipose macrophage infiltration is associated with insulin resistance and vascular endothelial dysfunction in obese subjects, Arterioscler Thromb Vasc Biol 28, 1654-1659.
18. Cinti, S., Mitchell, G., Barbatelli, G., Murano, I., Ceresi, E., Faloia, E., Wang, S., Fortier, M., Greenberg, A. S., and Obin, M. S. (2005) Adipocyte death defines macrophage localization and function in adipose tissue of obese mice and humans, J Lipid Res 46, 2347-2355.
19. Yamanaka, H., Moriguchi, T., Masuyama, N., Kusakabe, M., Hanafusa, H., Takada, R., Takada, S., and Nishida, E. (2002) JNK functions in the non-canonical Wnt pathway to regulate convergent extension movements in vertebrates, EMBO Rep 3, 69-75.
20. Hirosumi, J., Tuncman, G., Chang, L., Gorgun, C. Z., Uysal, K. T., Maeda, K., Karin, M., and Hotamisligil, G. S. (2002) A central role for JNK in obesity and insulin resistance, Nature 420, 333-336.
21. Kim, J. Y., van de Wall, E., Laplante, M., Azzara, A., Trujillo, M. E, Hofmann, S. M., Schraw, T., Durand, J. L., Li, H., Li, G., Jelicks, L. A., Mehler, M. F., Hui, D. Y., Deshaies, Y., Shulman, G. I., Schwartz, G. J., and Scherer, P. E. (2007) Obesity-associated improvements in metabolic profile through expansion of adipose tissue, J Clin Invest 117, 2621-2637.
22. Solinas, G., Vilcu, C., Neels, J. G., Bandyopadhyay, G. K., Luo, J. L., Naugler, W., Grivennikov, S., Wynshaw-Boris, A., Scadeng, M., Olefsky, J. M., and Karin, M. (2007) JNK1 in hematopoietically derived cells contributes to diet-induced inflammation and insulin resistance without affecting obesity, Cell Metab 6, 386-397.
23. Sabio, G., Das, M., Mora, A., Zhang, Z., Jun, S. Y., Ko, H. S., Barrett, T., Kim, J. K., and Davis, R. J. (2008) A stress signaling pathway in adipose tissue regulates hepatic insulin resistance, Science 322, 1539-1543.
24. Vallerie, S. N., Furuhashi, M., Fucho, R., and Hotamisligil, G. S. (2008) A predominant role for parenchymal c-Jun amino terminal kinase (JNK) in the regulation of systemic insulin sensitivity, PLoS One 3, e3151.
25. Li, S.-t., and Yuen, D. “Implant devices for nerve repair,” U.S. Pat. No. 6,716,225 (published Apr. 6, 2004).
26. Slepian, M. J., and Massia, S. P. “Local polymeric gel cellular therapy,” U.S. Pat. No. 6,290,729 (published Sep. 18, 2001).
27. Drake, C. F., and Arch, A. J. “Controlled release system,” United States (published May 6, 1986).
28. Satoh, W., Matsuyama, M., Takemura, H., Aizawa, S., and Shimono, A. (2008) Sfrp1, Sfrp2, and Sfrp5 regulate the Wnt/beta-catenin and the planar cell polarity pathways during early trunk formation in mouse, Genesis 46, 92-103.
29. Maeda, N., Takahashi, M., Funahashi, T., Kihara, S., Nishizawa, H., Kishida, K., Nagaretani, H., Matsuda, M., Komuro, R., Ouchi, N., Kuriyama, H., Hotta, K., Nakamura, T., Shimomura, I., and Matsuzawa, Y. (2001) PPARgamma ligands increase expression and plasma concentrations of adiponectin, an adipose-derived protein, Diabetes 50, 2094-2099.
30. Mano, T., Luo, Z., Suhara, T., Smith, R. C., Esser, S., and Walsh, K. (2000) Expression of wild-type and noncleavable Fas ligand by tetracycline-regulated adenoviral vectors to limit intimal hyperplasia in vascular lesions., Hum. Gene Ther. 11, 1625-1635.
31. Maeda, N., Shimomura, I., Kishida, K., Nishizawa, H., Matsuda, M., Nagaretani, H., Furuyama, N., Kondo, H., Takahashi, M., Arita, Y., Komuro, R., Ouchi, N., Kihara, S., Tochino, Y., Okutorni, K., Horie, M., Takeda, S., Aoyama, T., Funahashi, T., and Matsuzawa, Y. (2002) Diet-induced insulin resistance in mice lacking adiponectin/ACRP30, Nat. Med. 8, 731-737.
32. Shibata, R., Ouchi, N., Ito, M., Kihara, S., Shiojima, I., Pimentel, D. R., Kumada, M., Sato, K., Schiekofer, S., Ohashi, K., Funahashi, T., Colucci, W. S., and Walsh, K. (2004) Adiponectin-mediated modulation of hypertrophic signals in the heart, Nat. Med. 10, 1384-1389.
33. Bligh, E. G., and Dyer, W. J. (1959) A rapid method of total lipid extraction and purification, Can J Biochem Physiol 37, 911-917.
34. Gillies, S. D., Watkins, N. J., Baker, M. P., and Lo, K.-m. “Fc-interferon-beta fusion proteins,” U.S. Pat. No. 7,670,595 (published Mar. 2, 2010).

1. A method of reducing elevated glucose levels, comprising:
a) providing a subject with elevated glucose levels and a composition comprising at least a portion of human Sfrp5;
b) administering said composition to said subject; and
c) measuring said glucose levels of said subject until they are reduced.

2. The method of claim 1, wherein said subject is a human.

3. The method of claim 1, wherein said elevated glucose levels are reduced by at least 20%.

4. The method of claim 1, wherein said elevated glucose levels are reduced by at least 40%.

5. The method of claim 1, wherein said composition comprises a fusion protein comprising at least a portion of human Sfrp5, said portion comprising a portion of the sequence of SEQ ID NO: 2.

6. The method of claim 5, wherein said portion consists of at least one domain of human Sfrp5.

7. The method of claim 5, wherein said fusion protein comprises a poly-histidine tract.

8. The method of claim 5, wherein said fusion protein comprises at least a portion of an immunoglobulin molecule.

9. The method of claim 8, wherein said portion of an immunoglobulin molecule consists of an Fc fragment.

10. A method of reducing elevated glucose levels, comprising:
a) providing a subject with elevated glucose levels and a composition comprising an antibody or portion thereof reactive with human Wnt5a;
b) administering said composition to said subject; and
c) measuring said glucose levels of said subject until they are reduced.

11. The method of claim 10, wherein said antibody is a humanized monoclonal antibody reactive with human Wnt5a.

12. A method of measuring metabolic function, comprising:
a) providing i) a sample from a subject and ii) a reagent for measuring human Sfrp5 protein or human Sfrp5 nucleic acid;
b) measuring the level of Sfrp5 protein or nucleic acid as an indicator of metabolic function.

13. The method of claim 12, wherein said reagent is an antibody reactive with human Sfrp5 protein.

14. The method of claim 13, wherein said antibody is specific for human Sfrp5.

15. The method of claim 12, wherein said reagent is a probe for measuring human Sfrp5 mRNA.


SEQUENCE LISTING (35 sequences)

SEQ ID NO: 1 - protein, 314 aa, Mus musculus
MWVAWSARTAALALLLGALHGAPTRGQEYDYYGWQAEPLHGRSYSKPPQCIDIPADLPLCHTVGYKRMRLPNLLEHESLAEVKQQASSWLPLLAKRCHSDTQVFLCSLFAPVCLDRPIYPCRSLCEAVRAGCAPLMEAYGFPWPEMLHCHKFPLDNDLCIAVQFGHLPATAPPVTKICAQCEMEHSADGLMEQMCSSDFVVKMRIKEIKIDNGDRKLIGAQKKKKLLKAGPLKRKDTKKLVLHMKNGASCPCPQLDNLTGSFLVMGRKVEGQLLLTAVYRWDKKNKEMKFAVKFMFSYPCSLYYPFFYGAAEPH

SEQ ID NO: 2 - protein, 317 aa, Homo sapiens
MRAAAAGGGVRTAALALLLGALHWAPARCEEYDYYGWQAEPLHGRSYSKPPQCLDIPADLPLCHTVGYKRMRLPNLLEHESLAEVKQQASSWLPLLAKRCHSDTQVFLCSLFAPVCLDRPIYPCRSLCEAVRAGCAPLMEAYGFPWPEMLHCHKFPLDNDLCIAVQFGHLPATAPPVTKICAQCEMEHSADGLMEQMCSSDFVVKMRIKEIKIENGDRKLIGAQKKKKLLKPGPLKRKDTKRLVLHMKNGAGCPCPQLDSLAGSFLVMGRKVDGQLLLMAVYRWDKKNKEMKFAVKFMFSYPCSLYYPFFYGAAEPH

SEQ ID NO: 3 - DNA, 1883 bp, Homo sapiens
agtcggggcgcccgcagcgcaggctgccacccacctgggcgacctccgcggcggcggcggcggcggctgggtagagtcagggccgggggcgcacgccggaacacctgggccgccgggcaccgagcgtcggggggctgcgcggcgcgcacctggagagggcgcagccatgcgggcggcggcggcgggggggggcgtgcggacggccgcgctggcgctgctgctgggggcgctgcactgggcgccggcgcgctgcgaggagtacgactactatggctggcaggccgagccgctgcacggccgctcctactccaagccgccgcagtgccttgacatccctgccgacctgccgctctgccacacggtgggctacaagcgcatgcggctgcccaacctgctggagcacgagagcctggccgaagtgaagcagcaggcgagcagctggctgccgctgctggccaagcgctgccactcggatacgcaggtcttcctgtgctcgctctttgcgcccgtctgtctcgaccggcccatctacccgtgccgctcgctgtgcgaggccgtgcgcgccggctgcgcgccgctcatggaggcctacggcttcccctggcctgagatgctgcactgccacaagttccccctggacaacgacctctgcatcgccgtgcagttcgggcacctgcccgccaccgcgcctccagtgaccaagatctgcgcccagtgtgagatggagcacagtgctgacggcctcatggagcagatgtgctccagtgactttgtggtcaaaatgcgcatcaaggagatcaagatagagaatggggaccggaagctgattggagcccagaaaaagaagaagctgctcaagccgggccccctgaagcgcaaggacaccaagcggctggtgctgcacatgaagaatggcgcgggctgcccctgcccacagctggacagcctggcgggcagcttcctggtcatgggccgcaaagtggatggacagctgctgctcatggccgtctaccgctgggacaagaagaataaggagatgaagtttgcagtcaaattcatgttctcctacccctgctccctctactaccctttcttctacggggcggcagagccccactgaagggcactcctccttgccctgccagctgtgccttgcttgccctctggccccgccccaacttccaggctgacccggccctactggagggtgttttcacgaatgttgttactggcacaaggcctaagggatgggcacggagcccaggctgtcctttttgacccaggggtcctggggtccctgggatgttgggcttcctctctcaggagcagggcttcttcatctgggtgaagacctcagggtctcagaaagtaggcaggggaggagagggtaagggaaaggtggaggggctcagggcaccctgaggcggaggtttcagagtagaaggtggtgtcagctccagctcccctctgtcggtggtggggcctcaccttgaagagggaagtctcaatattaggctaagctatttgggaaagttctccccaccgcccctgtacgcgtcatcctagccccccttaggaaaggagttagggtctcagtgcctccagccacaccccctgccttccccagcttgcccatttccctgccccaaggcccagagctccccccagactggagagcaagcccagcccagcctcggcatagacccccttctggtccgcccgcggctcgattcccgggattcattcctcagcctctgcttctcccttttatcccaataagttattgctactgctgtgaggccataggtactagacaaccaatacatgcagggttgggttttctaatttttttaactttttaattaaatcaaagaaaacaataa

SEQ ID NO: 4 - protein, 381 aa, Homo sapiens
PLQKSIGILSPGVALGMAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 5 - protein, 18 aa, Artificial Sequence
  Synthetic
WAPARCEEYDYYGWQAEP

SEQ ID NO: 6 - protein, 10 aa, Artificial Sequence
  Synthetic
PKDLPRDWLW

SEQ ID NO: 7 - protein, 17 aa, Artificial Sequence
  Synthetic
APTRGQEYDYYGWQAEP

SEQ ID NO: 8 - protein, 19 aa, Artificial Sequence
  Synthetic
VKMRIKEIKIDNGDRKLIG

SEQ ID NO: 9 - DNA, 22 bp, Artificial Sequence
  Synthetic
gaaagttgattggagcccagaa

SEQ ID NO: 10 - DNA, 22 bp, Artificial Sequence
  Synthetic
gcccgtcaggttgtctaactgt

SEQ ID NO: 11 - DNA, 20 bp, Artificial Sequence
  Synthetic
cagccagatgcagttaacgc

SEQ ID NO: 12 - DNA, 23 bp, Artificial Sequence
  Synthetic
gcctactcattgggatcatcttg

SEQ ID NO: 13 - DNA, 22 bp, Artificial Sequence
  Synthetic
ctttggctatgggcttccagtc

SEQ ID NO: 14 - DNA, 24 bp, Artificial Sequence
  Synthetic
gcaaggaggacagagtttatcgtg

SEQ ID NO: 15 - DNA, 20 bp, Artificial Sequence
  Synthetic
cttctgctgtggaaatgcaa

SEQ ID NO: 16 - DNA, 20 bp, Artificial Sequence
  Synthetic
agaggggctggtaggttgat

SEQ ID NO: 17 - DNA, 17 bp, Artificial Sequence
  Synthetic
tgccatccatgcggaaa

SEQ ID NO: 18 - DNA, 20 bp, Artificial Sequence
  Synthetic
agcgggaagaactcctcttc

SEQ ID NO: 19 - DNA, 20 bp, Artificial Sequence
  Synthetic
atacaggtgccaggaaggtg

SEQ ID NO: 20 - DNA, 20 bp, Artificial Sequence
  Synthetic
caagggcagaaagttggtgt

SEQ ID NO: 21 - DNA, 16 bp, Artificial Sequence
  Synthetic
aggttggatggcaggc

SEQ ID NO: 22 - DNA, 22 bp, Artificial Sequence
  Synthetic
gtctcacccttaggaccaagaa

SEQ ID NO: 23 - DNA, 20 bp, Artificial Sequence
  Synthetic
ttgggtcagcactggctctg

SEQ ID NO: 24 - DNA, 20 bp, Artificial Sequence
  Synthetic
tggcggtgtgcagtgctatc

SEQ ID NO: 25 - DNA, 20 bp, Artificial Sequence
  Synthetic
gatgttccccattgaggccg

SEQ ID NO: 26 - DNA, 20 bp, Artificial Sequence
  Synthetic
gtttcaggtcatcaggccgc

SEQ ID NO: 27 - DNA, 20 bp, Artificial Sequence
  Synthetic
acctattcctgcgtcggtgt

SEQ ID NO: 28 - DNA, 20 bp, Artificial Sequence
  Synthetic
gcatcgaagaccgtgttctc

SEQ ID NO: 29 - DNA, 23 bp, Artificial Sequence
  Synthetic
gtcctgtcctcagatgaaattgg

SEQ ID NO: 30 - DNA, 24 bp, Artificial Sequence
  Synthetic
gcagggtcaagagtagtgaaggtt

SEQ ID NO: 31 - DNA, 22 bp, Artificial Sequence
  Synthetic
aatcaagaacgaaagtcggagg

SEQ ID NO: 32 - DNA, 19 bp, Artificial Sequence
  Synthetic
gcgggtcatgggaataacg

SEQ ID NO: 33 - DNA, 19 bp, Artificial Sequence
  Synthetic
gctccaagcagatgcagca

SEQ ID NO: 34 - DNA, 18 bp, Artificial Sequence
  Synthetic
ccggatgtgaggcagcag

SEQ ID NO: 35 - protein, 317 aa, Artificial Sequence
  Synthetic
  misc_feature (2)..(3): Xaa can be any naturally occurring amino acid
  misc_feature (5)..(6): Xaa can be any naturally occurring amino acid
  misc_feature (10)..(10): Xaa can be any naturally occurring amino acid
  misc_feature (24)..(24): Xaa can be any naturally occurring amino acid
  misc_feature (27)..(27): Xaa can be any naturally occurring amino acid
  misc_feature (29)..(30): Xaa can be any naturally occurring amino acid
  misc_feature (214)..(214): Xaa can be any naturally occurring amino acid
  misc_feature (232)..(232): Xaa can be any naturally occurring amino acid
  misc_feature (242)..(242): Xaa can be any naturally occurring amino acid
  misc_feature (252)..(252): Xaa can be any naturally occurring amino acid
  misc_feature (260)..(260): Xaa can be any naturally occurring amino acid
  misc_feature (262)..(262): Xaa can be any naturally occurring amino acid
  misc_feature (273)..(273): Xaa can be any naturally occurring amino acid
  misc_feature (279)..(279): Xaa can be any naturally occurring amino acid
MXXAXXGGGXRTAALALLLGALHXAPXRXXEYDYYGWQAEPLHGRSYSKPPQCLDIPADLPLCHTVGYKRMRLPNLLEHESLAEVKQQASSWLPLLAKRCHSDTQVFLCSLFAPVCLDRPIYPCRSLCEAVRAGCAPLMEAYGPPWPEMLHCHKFPLDNDLCIAVQFGHLPATAPPVTKICAQCEMEHSADGLMEQMCSSDFVVKMRIKEIKIXNGDRKLIGAQKKKKLLKXGPLKRKDTKXLVLHMKNGAXCPCPQLDXLXGSFLVMGRKVXGQLLLXAVYRWDKKNKEMKFAVKFMFSYPCSLYYPFFYGAAEPH